(19) United States
(12) Patent Application Publication
(10) Pub. No.: US 2024/0108347 A1
(43) Pub. Date: Apr. 4, 2024

(54) Surgical Staplers and Related Methods

(71) Applicant: Franklin Institute of Innovation, LLC, Peachtree City, GA (US)

(72) Inventors: James S. Franklin, SR., Peachtree City, GA (US); Emily Williams, Atlanta, GA (US); Christian Sorensen, Alpharetta, GA (US); John Tipton, Atlanta, GA (US); Jason Weiss, Atlanta, GA (US)

(21) Appl. No.: 18/534,926

(22) Filed: Dec. 11, 2023

Related U.S. Application Data
(63) Continuation of application No. 17/054,382, filed on Nov. 10, 2020.
(60) Provisional application No. 62/669,798, filed on May 10, 2018.

(51) Int. Cl. A61B 17/115; A61B 17/00; A61B 17/32; A61B 17/34; A61B 90/00

(52) U.S. Cl. CPC A61B 17/1155; A61B 17/00234; A61B 17/320016; A61B 17/3474; A61B 90/361; A61B 2017/00292; A61B 2017/00557

(57) ABSTRACT

Surgical staplers and methods of their use are provided. In one embodiment, a surgical stapler includes a tubular shaft having a proximal end and a distal end, a handle extending from the proximal end of the tubular shaft, a head assembly extending from the distal end of the tubular shaft, and an insertion guide configured to extend through the head assembly. The insertion guide includes an expandable member configured to move between a collapsed configuration and an expanded configuration and a camera configured to be positioned at least partially within the expandable member.

### CROSS-REFERENCE TO RELATED APPLICATIONS

This is a continuation of U.S. application Ser. No. 17/054,382, filed Nov. 10, 2020, which is a U.S. national stage of PCT/US2019/031757, filed May 10, 2019, which claims the benefit of priority to U.S. Provisional Patent Application No. 62/669,798, filed May 10, 2018, all of which are incorporated herein by reference.

### TECHNICAL FIELD

The present disclosure relates generally to surgical instruments and methods, and more particularly to surgical staplers and related methods of using such staplers to perform an end-to-end anastomosis between two tubular tissue segments.

### BACKGROUND

Various types of surgical procedures may necessitate anastomosis between two tubular tissue segments in order to restore the natural function of the tissue segments. For example, during colon and rectal resection surgery, a diseased or otherwise defective portion of the colon of a patient may be removed, and the colon segments above and below the removed portion may need to be reconnected to restore the natural flow through the colon. Traditionally, the upper and lower colon segments were rejoined by suturing the respective ends of the colon segments to one another. In recent years, surgical staplers have been developed for performing an end-to-end anastomosis between two tubular tissue segments, allowing clinicians to reconnect the tissue segments in a more efficient and reliable manner during a surgical procedure.

Existing surgical staplers for performing an end-to-end anastomosis generally may include an elongated tubular shaft, a handle attached to a proximal end of the tubular shaft, and a head assembly attached to a distal end of the tubular shaft. The head assembly may include an outer shell that contains a mechanism for forming a circular array of staples to connect two tubular tissue segments. For example, a staple guide may be fixedly positioned within the outer shell along a distal end thereof, and a staple pusher may be movably positioned within the outer shell and configured for advancing a plurality of staples through the staple guide. The head assembly also may include a mechanism for coring respective portions of the tissue segments being stapled to one another. For example, a circular knife may be movably positioned within the outer shell and configured for cutting inner portions of the tissue segments within the circular array of staples. The formation of the array of staples and the removal of the inner portions of the tissue segments may be facilitated by an anvil that is removably attached to a trocar of the head assembly. The trocar may be movably positioned within the outer shell and configured to draw an anvil head of the anvil adjacent to the distal end of the outer shell. In this manner, the staples may be advanced through the staple guide, through respective portions of the tissue segments, and against the anvil head to facilitate desired deformation of the staples. Additionally, the circular knife may be advanced through respective portions of the tissue segments and against the anvil head to cut and remove inner portions of the tissue segments within the circular array of staples.

When existing surgical staplers are used to perform an end-to-end anastomosis between two colon segments, the head assembly and a portion of the tubular shaft of the stapler may be inserted through the anus of the patient and advanced through the rectum to the end of the lower color segment, while the handle remains outside of the patient to allow the clinician to control positioning and operation of the stapler. The head assembly generally may be relatively large in order to accommodate the various components for staple formation and for cutting the inner portions of the colon segments being joined. Further, the distal end face of the head assembly may be flat or relatively flat in order to cooperate with the mating portion of the anvil head, resulting in an abrupt edge along the outer circumference of the head assembly. In many instances, it may be challenging for the clinician to insert the head assembly through the anus and the rectum and advance the head assembly to the desired location at the end of the lower colon segment. For example, due to the size and/or shape of the head assembly, it may be difficult to advance the head assembly through the anus and then through the contours of the rectum without snagging on the mucosa folds. In some instances, as the head assembly is advanced to the desired location, the size and/or shape of the head assembly may result in injury to the surrounding anatomy and various complications for the patient. For example, introduction of the head assembly may result in anal sphincter injury, which may lead to loss of voluntary control of bowel function. Additionally, as the head assembly is moved to the desired location, it may engage and cause damage to the internal lining of the colon, which may lead to bleeding or potentially a leak along the respective region of the colon. Finally, in view of the known challenges in advancing the head assembly to the desired location, clinicians often may remove a larger portion of the colon than is truly needed in order to ease insertion and positioning of the head assembly within the patient.

There remains a need for improved surgical staplers and methods of using such staplers to perform an end-to-end anastomosis between two tubular tissue segments, such as colon segments following resection of a portion of the colon. In particular, it would be advantageous to provide a surgical stapler that eases insertion and advancement of a head assembly of the stapler to a desired location for performing the anastomosis, while reducing incidence of injury to the surrounding anatomy and complications for the patient.

### BRIEF SUMMARY

Surgical staplers and methods of using such staplers to perform an end-to-end anastomosis between two tubular tissue segments are provided. According to one aspect, a surgical stapler is provided. In one embodiment, the surgical stapler includes a tubular shaft having a proximal end and a distal end, a handle extending from the proximal end of the tubular shaft, a head assembly extending from the distal end of the tubular shaft, and an insertion guide configured to extend through the head assembly. The insertion guide includes an expandable member configured to move between a collapsed configuration and an expanded configuration and a camera configured to be positioned at least partially within the expandable member.

In another embodiment, the surgical stapler includes a tubular shaft having a proximal end and a distal end, a handle extending from the proximal end of the tubular shaft, a head assembly extending from the distal end of the tubular shaft, and an insertion guide. The insertion guide is configured to extend through the head assembly. The insertion guide includes an expandable member and a tube extending from a proximal end of the expandable member. The expandable member is configured to move between a collapsed configuration and an expanded configuration. The tube includes a first lumen and a second lumen extending therethrough.

According to another aspect, a surgical stapler system is provided. In one embodiments, the surgical stapler system includes a surgical stapler, a fluid delivery device, a camera module, and an air delivery device. The surgical stapler includes a tubular shaft having a proximal end and a distal end, a handle extending from the proximal end of the tubular shaft, a head assembly extending from the distal end of the tubular shaft, and an insertion guide configured to extend through the head assembly. The insertion guide includes an expandable member, a camera, and a tube. The expandable member is configured to move between a collapsed configuration and an expanded configuration. The camera is configured to be positioned at least partially within the expandable member. The tube extends from a proximal end of the expandable member. The tube includes a first lumen, a second lumen, and a third lumen extending therethrough. The fluid delivery device is configured to attach to the tube and be in fluid communication with the first lumen. The camera module is configured to attach to the tube and be in operable communication with the camera. The air delivery device is configured to attach to the tube and be in fluid communication with the third lumen.

According to still another aspect, a method of introducing a surgical stapler into a patient is provided. In one embodiment, the method includes the steps of advancing an insertion guide through a head assembly of the surgical stapler, such that the insertion guide extends distally beyond a distal end face of the head assembly, moving an expandable member of the insertion guide from a collapsed configuration to an expanded configuration, inserting the expandable member and the head assembly into a body cavity of the patient while the expandable member is in the expanded configuration, and capturing, via a camera of the insertion guide, video or image data of internal anatomy of the patient.

In another embodiment, the method includes the steps of advancing an insertion guide through a head assembly of the surgical stapler, such that the insertion guide extends distally beyond a distal end face of the head assembly, moving an expandable member of the insertion guide from a collapsed configuration to an expanded configuration, inserting the expandable member and the head assembly into a body cavity of the patient while the expandable member is in the expanded configuration, and delivering air through the insertion guide to insufflate the body cavity.

These and other aspects and embodiments of the present disclosure will be apparent or will become apparent to one of ordinary skill in the art upon review of the following detailed description when taken in conjunction with the drawings and the appended claims.

### BRIEF DESCRIPTION OF THE DRAWINGS

FIG. 1A is a perspective view of a surgical stapler in accordance with one or more embodiments of the disclosure, showing a tubular shaft, a handle, a head assembly, a trocar, an anvil, and an insertion guide of the surgical stapler.

FIG. 1B is a detailed side view of a portion of the surgical stapler of FIG. 1A, showing the trocar in a retracted position.

FIG. 1C is a detailed side view of a portion of the surgical stapler of FIG. 1A, showing the trocar in an extended position.

FIG. 1D is a detailed side view of a portion of the surgical stapler of FIG. 1A, showing the anvil attached to the trocar, with the trocar in the extended position.

FIG. 1E is a detailed side view of a portion of the surgical stapler of FIG. 1A, showing the anvil attached to the trocar, with the trocar in the retracted position.

FIG. 1F is a detailed side view of a portion of the surgical stapler of FIG. 1A, showing the insertion guide extending through the head assembly, with an expandable member of the insertion guide in a collapsed configuration.

FIG. 1G is a detailed side view of a portion of the surgical stapler of FIG. 1A, showing the insertion guide extending through the head assembly, with the expandable member in an expanded configuration.

FIG. 1H is a detailed side view of a portion of the surgical stapler of FIG. 1A, showing the insertion guide extending through the tubular shaft and the head assembly, with an expandable member of the insertion guide in a collapsed configuration.

FIG. 1I is a detailed side view of a portion of the surgical stapler of FIG. 1A, showing the insertion guide extending through the tubular shaft and the head assembly, with the expandable member in an expanded configuration.

FIGS. 2A-2H illustrate a method of using the surgical stapler of FIG. 1A to perform an end-to-end anastomosis between two tissue segments of a tubular tissue structure in accordance with one or more embodiments of the disclosure FIG. 2A is a cross-sectional side view of the tubular tissue structure, showing a portion of the tubular tissue structure to be removed.

FIG. 2B is a partial cross-sectional side view of a first tissue segment and a second tissue segment of the tubular tissue structure, showing the anvil of the surgical stapler of FIG. 1A positioned partially within and extending from the first tissue segment.

FIG. 2C is a partial cross-sectional side view of the first tissue segment and the second tissue segment, showing the expandable member of the surgical stapler of FIG. 1A in the expanded configuration and positioned within the second tissue segment adjacent a closed end thereof.

FIG. 2D is a partial cross-sectional side view of the first tissue segment and the second tissue segment, showing the head assembly of the surgical stapler of FIG. 1A positioned adjacent the closed end of the second tissue segment, with the trocar in the retracted position.

FIG. 2E is a partial cross-sectional side view of the first tissue segment and the second tissue segment, showing the head assembly positioned adjacent the closed end of the second tissue segment, with the trocar in the extended position and extending through the closed end of the second tissue segment.

FIG. 2F is a partial cross-sectional side view of the first tissue segment and the second tissue segment, showing the trocar in the extended position and attached to the anvil.

FIG. 2G is a partial cross-sectional side view of the first tissue segment and the second tissue segment, showing the trocar in the retracted position and attached to the anvil.

FIG. 2H is a cross-sectional side view of the first tissue segment and the second tissue segment, showing the first tissue segment and the second tissue segment joined to one another by a circular array of staples.

FIG. 3A is a side view of an insertion guide as may be used with the surgical stapler of FIG. 1A in accordance with one or more embodiments of the disclosure, showing a fluid delivery device, a camera module, and an air delivery device connected to the insertion guide.

FIG. 3B is a detailed end view of a portion of the insertion guide of FIG. 3A, taken along line 3B-3B of FIG. 3A, showing a first lumen, a second lumen, and a third lumen of the insertion guide and a camera positioned within the second lumen.

FIG. 3C is a detailed cross-sectional end view of a portion of the insertion guide of FIG. 3A, taken along line 3C-3C of FIG. 3A, showing the first lumen, the second lumen, and the third lumen of the insertion guide.

FIG. 3D is a detailed side view of a portion of the surgical stapler of FIG. 1A, showing the insertion guide of FIG. 3A extending through the head assembly, with an expandable member of the insertion guide in a collapsed configuration.

FIG. 3E is a detailed side view of a portion of the surgical stapler of FIG. 1A, showing the insertion guide of FIG. 3A extending through the head assembly, with the expandable member in an expanded configuration.

FIG. 3F is a detailed side view of a portion of the surgical stapler of FIG. 1A, showing the insertion guide of FIG. 3A extending through the tubular shaft and the head assembly, with an expandable member of the insertion guide in a collapsed configuration.

FIG. 3G is a detailed side view of a portion of the surgical stapler of FIG. 1A, showing the insertion guide of FIG. 3A extending through the tubular shaft and the head assembly, with the expandable member in an expanded configuration.

### DETAILED DESCRIPTION

Improved surgical staplers and methods have been developed for performing an end-to-end anastomosis between two tubular tissue segments, such as colon segments following resection of a portion of the colon. Such surgical staplers and methods advantageously may ease insertion and advancement of a head assembly of a stapler to a desired location for performing an anastomosis, while reducing incidence of injury to the surrounding anatomy and corresponding complications for the patient. In particular, the surgical staplers described herein may include an insertion guide having an atraumatic expandable member, such as a balloon, that is configured to be positioned about a distal end of the head assembly. The expandable member may be moved, or transitioned, from a collapsed configuration to an expanded configuration prior to introduction of the stapler into the patient. While in the expanded configuration, the expandable member may cover a distal end face of the head assembly, thereby inhibiting the distal end face from engaging the surrounding anatomy as the head assembly is advanced toward a desired location within a tissue segment. Once the expandable member reaches the desired location, the expandable member may be moved, or transitioned, from the expanded configuration to the collapsed configuration, and the insertion guide may be withdrawn from the head assembly and removed from the patient.

In preferred embodiments, the insertion guide is entirely removable from the stapler such that after the expandable member (e.g., balloon) in the expanded configuration (e.g., inflated) has been used to deliver the stapler into the patient, the expandable member can be collapsed and the insertion guide then removed entirely from the stapler to ensure that the balloon and insertion guide are completely out of the way for the anastomosis to be securely carried out.

As compared to existing staplers for performing an end-to-end anastomosis, the surgical staplers described herein may allow clinicians to more easily and efficiently introduce a stapler into a patient in a manner that inhibits injury to the surrounding anatomy. For example, in the context of rejoining an upper colon segment and a lower colon segment following resection of a portion of the colon, the atraumatic expandable member of the insertion guide may ease insertion of the head assembly through the anus and advancement of the head assembly through the rectum to the closed end of the lower colon segment. In particular, the expandable member may engage portions of the surrounding anatomy instead of the head assembly, and the curved shape and flexible nature of the expandable member may facilitate advancement of the head assembly through restricted regions and contours of the anatomy without causing injury to the patient. As a result, the surgical staplers described herein may reduce incidence of anal sphincter injury and/or damage to the internal lining of the colon, as may be experienced during use of existing surgical staplers. Further, the surgical staplers described herein may obviate the clinical practice of removing a larger portion of the colon than is needed for the purpose of easing insertion and positioning of the stapler.

Surgical Staplers

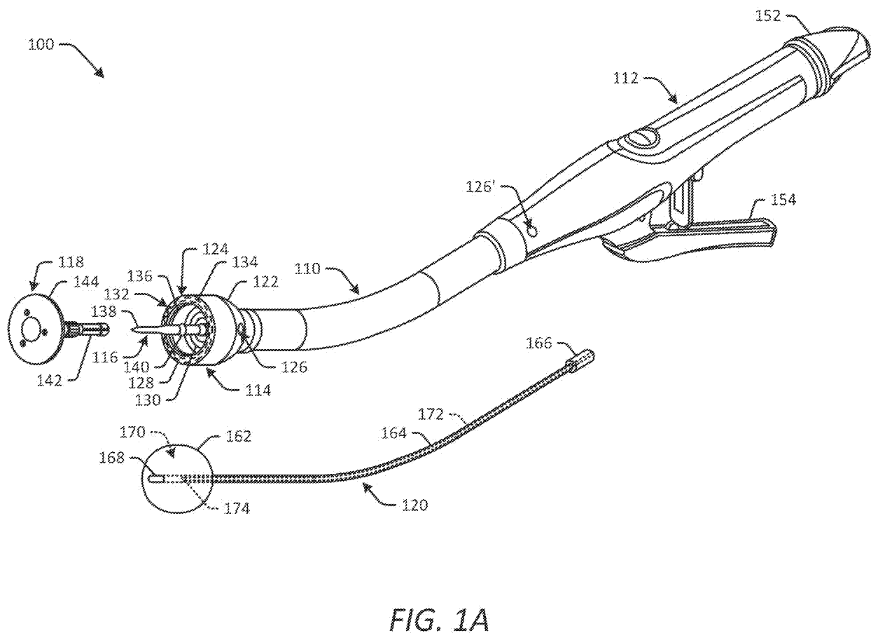

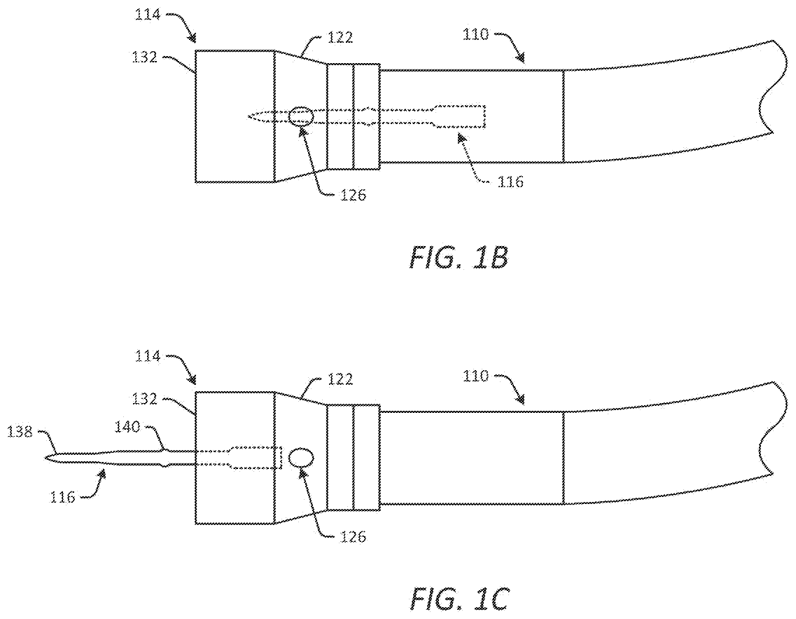

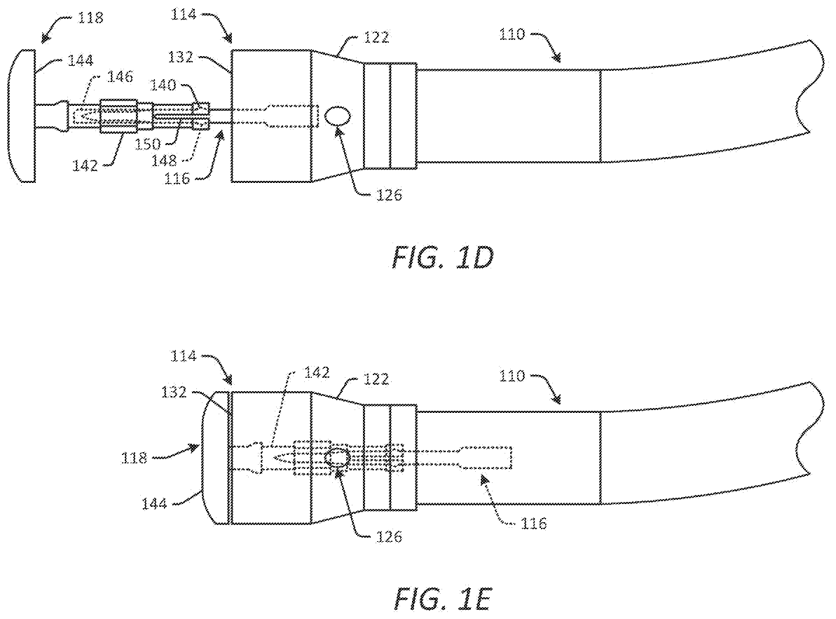

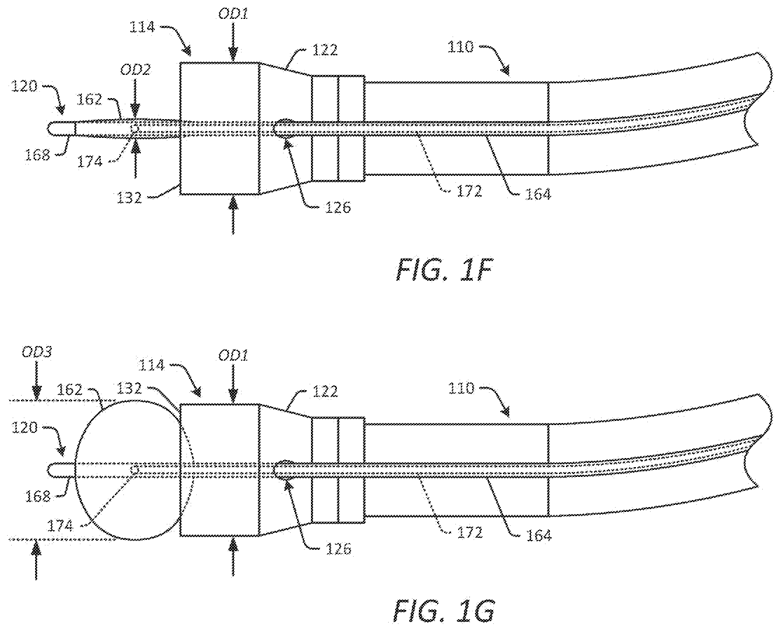

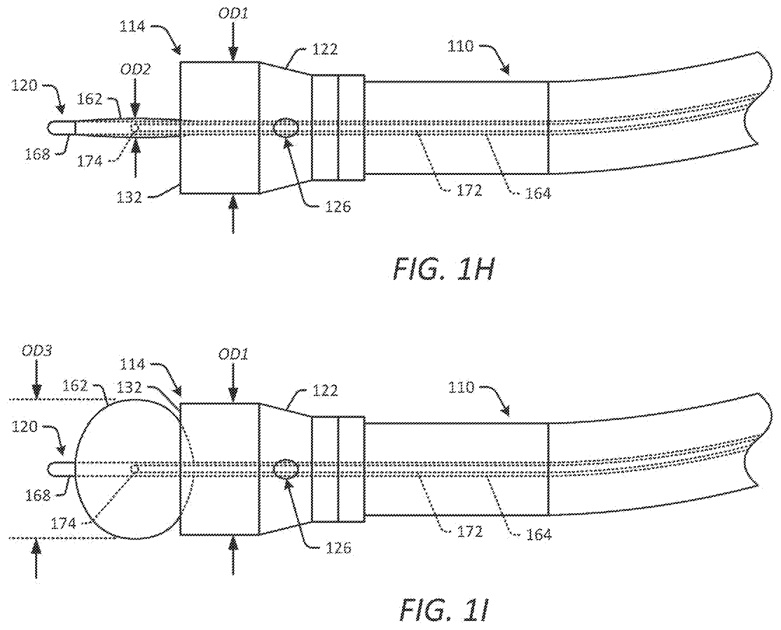

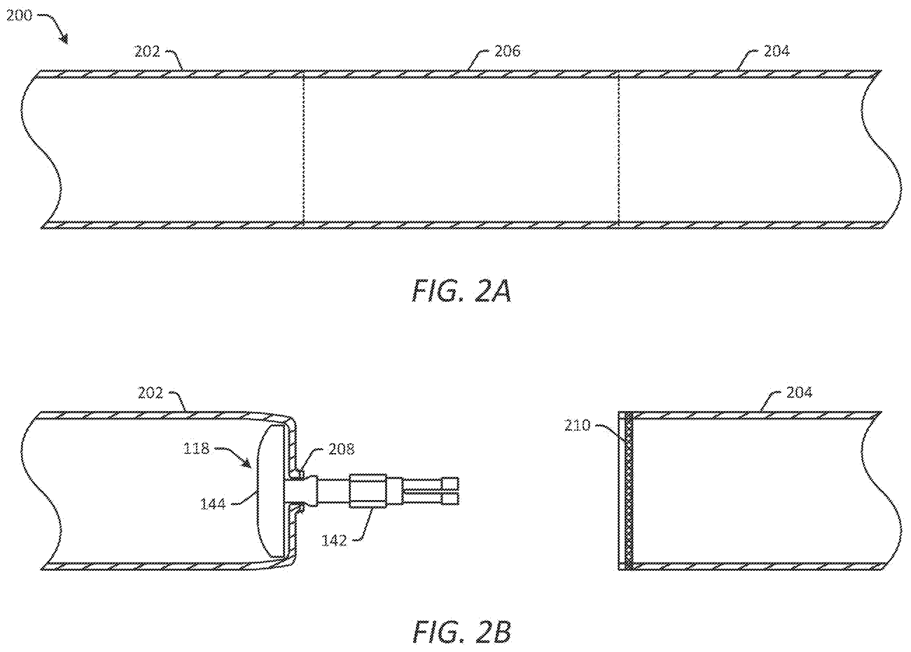

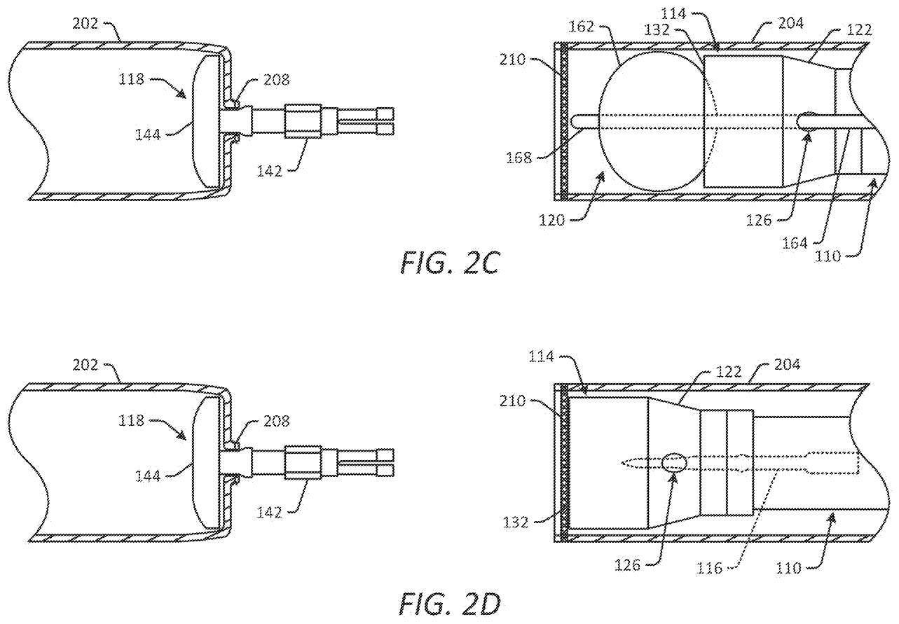

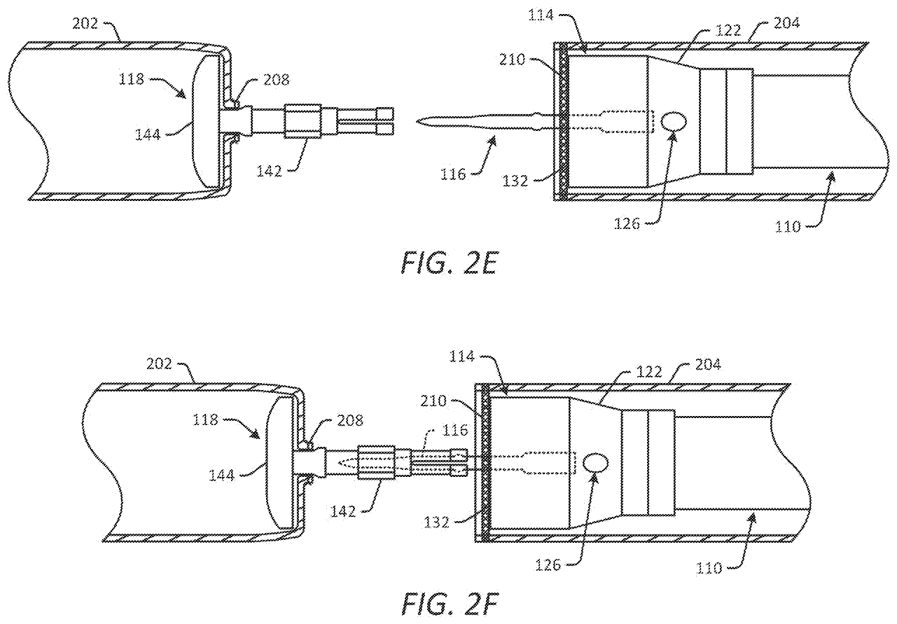

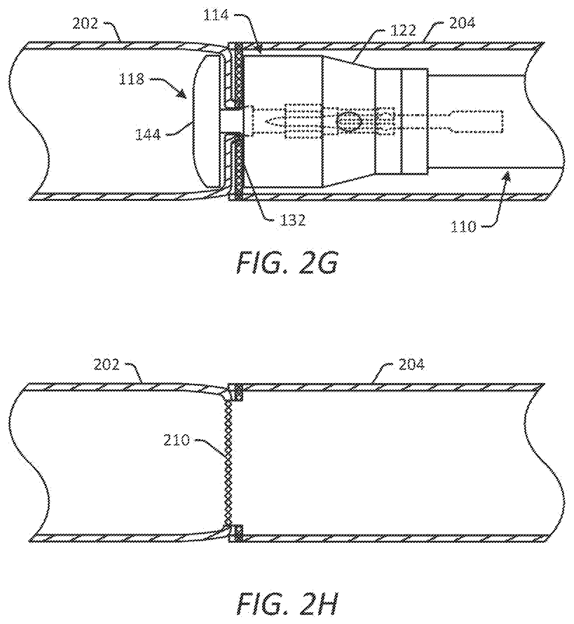

FIGS. 1A-1G illustrate a surgical stapler 100 (which also may be referred to as an “circular stapler” or simply a “stapler”) configured to perform an end-to-end anastomosis between two tubular tissue segments of a patient, in accordance with one or more embodiments of the disclosure. For example, the surgical stapler 100 may be used following resection of a portion of a patient's colon to reconnect upper and lower colon segments, as described below with respect to FIGS. 2A-2H. As shown in FIG. 1A, the surgical stapler 100 includes a tubular shaft 110, a handle 112, a head assembly 114, a trocar 116, an anvil 118, and an insertion guide 120. During use of the surgical stapler 100, the head assembly 114 and a portion of the tubular shaft 110 may be inserted into a patient, while the handle 112 remains outside of the patient to allow a clinician to control operation of the stapler 100. As described below, the insertion guide 120 may be used to facilitate insertion of the head assembly 114 into the patient and to guide advancement of the head assembly 114 to a target location for performing an anastomosis. Once the head assembly 114 is positioned at or near the target location, the insertion guide 120 may be removed from the patient, and the head assembly 114, the trocar 116, and the anvil 118 then may be used in a conventional manner to connect two tubular tissue segments via a circular array of staples.

In some preferred embodiments, the insertion guide and/or the expandable member are readily separable from the tubular shaft, handle, and head assembly, so that the insertion guide and/or the expandable member can be easily replaced during use of the surgical stapler. For example, if the expandable member were to tear or otherwise fail before or during insertion of the head assembly into the patient, then the surgeon could quickly replace the insertion guide and/or the expandable member and resume the procedure. In a preferred embodiment, the insertion guide is a single component assembly comprising the expandable member, tube, port, and distal tip. In this way, the entire insertion guide can be rapidly removed from the shaft and replaced with a new insertion guide.

In an alternative embodiment, the insertion guide includes a distal portion that includes the expandable member and a proximal portion that are releasably connected together. The connector may be snap-lock connector, a frictionally engagement (e.g., overlap) connector, a Luer-Lock type connector, or the like. In such an embodiment, the distal end portion is replaceable if needed, such that that part of the insertion guide can be replaced. In this way, the entire insertion guide need not be replaced, for example, if the balloon fails, and this partial replacement beneficially may permit the proximal end of the insertion guide to remain connected to a fluid source (used for filling the balloon) during the partial replacement, thus allowing the surgeon to quickly remove/replace the balloon portion.

The tubular shaft 110 may be formed as elongated hollow member having a proximal end and a distal end. As shown, the handle 112 may extend from the proximal end of the tubular shaft 110, and the head assembly 114 may extend from the distal end of the tubular shaft 110. In some embodiments, the handle 112 and the head assembly 114 may be fixedly attached to the tubular shaft 110. In other embodiments, the handle 112 and/or the head assembly 114 may be removably attached to the tubular shaft 110. As shown, the tubular shaft 110 may have a contoured shape including one or more curved regions and one or more straight regions. Alternatively, the tubular shaft 110 may have a straight shape extending from the proximal end to the distal end thereof. As described below, various internal components of the surgical stapler 100 may be positioned within or extend through the lumen of the tubular shaft 110 to allow components of the head assembly 114 to be controlled by the clinician from outside of the patient during use of the stapler 100. It will be appreciated that the illustrated tubular shaft 110 is merely one example embodiment, and that other shapes and configurations of the tubular shaft 110 may be used with the surgical stapler 100.

The handle 112 may be formed as an elongated member extending proximally from the proximal end of the tubular shaft 110. As shown, the handle 112 may have a contoured shape to allow the clinician to easily grasp the handle 112 and move the surgical stapler 100 relative to the patient. In some embodiments, a portion or all of the handle 112 may be formed as a hollow member defining an interior space for containing various internal components of the surgical stapler 100 therein. It will be appreciated that the illustrated handle 112 is merely one example embodiment, and that other shapes and configurations of the handle 112 may be used with the surgical stapler 100.

The head assembly 114 may be formed as an elongated assembly extending distally from the distal end of the tubular shaft 110. As shown, the head assembly 114 may include an outer shell 122 that is attached to a distal end portion of the tubular shaft 110 and configured to contain other components of the head assembly 114 therein. The outer shell 122 may be formed as a substantially hollow member including a central bore 124 defined therein. The central bore 124 may extend from a distal end toward a proximal end of the outer shell 122. In this manner, the central bore 124 may be in communication with the lumen of the tubular shaft 110 to allow various components of the surgical stapler 100 to extend through the tubular shaft 110 and the head assembly 114. As shown, the outer shell 122 also may include a lateral opening 126 that is defined in an outer surface of the outer shell 122 and in communication with the central bore 124. In other words, the lateral opening 126 may extend from the outer surface of the outer shell 122 to the central bore 124. As described below, the lateral opening 126 may allow the insertion guide 120 to pass through the lateral opening 126 and into the central bore 124 during use of the insertion guide 120.

The head assembly 114 may include one or more components to facilitate formation of a circular array of staples to connect two tubular tissue segments. For example, the head assembly 114 may include a staple guide 128 and a staple pusher 130. The staple guide 128 may be fixedly positioned within the central bore 124 of the outer shell 122. As shown, the staple guide 128 may extend to the distal end of the outer shell 122. In this manner, the distal end of the staple guide 128 and the distal end of the outer shell 122 may define a distal end face 132 of the head assembly 114. The distal end face 132 may be a planar or substantially planar surface extending transverse to, such as perpendicular to, the longitudinal axis of the head assembly 114. As shown, the staple guide 128 may be formed as a ring-shaped member having a plurality of slots 134 defined therein and a central aperture extending through the staple guide 128. The slots 134 may be arranged in a circular array, and each slot 134 may be configured to receive a respective staple therein. In this manner, the slots 134 of the staple guide 128 may maintain the plurality of staples in respective positions around the longitudinal axis of the head assembly 114 to facilitate formation of a circular array of staples for joining the tubular tissue segments. The staple pusher 130 may be movably positioned within the central bore 124 of the outer shell 122. For example, the staple pusher 130 may be configured to translate relative to the outer shell 122 along the longitudinal axis of the head assembly 114 between a retracted position and an extended position. As shown, the staple pusher 130 may be positioned axially between the staple guide 128 and the outer shell 122. The staple pusher 130 may include a base portion and a plurality of protrusions extending distally from the base portion and corresponding to the slots 134 of the staple guide 128. When the staple pusher 130 moves from the retracted position to the extended position, the protrusions of the staple pusher 130 may engage the respective slots 134 and cause the staples to be ejected therefrom. In some embodiments, the staple pusher 130 may include an aperture that is aligned with the lateral opening 126 of the outer shell 122 when the staple pusher 130 is in the retracted position. In this manner, the insertion guide 120 may pass through the lateral opening 126, through the aperture of the staple pusher 130, and into the central bore 124 during use of the insertion guide 120.

The head assembly 114 also may include one or more components for coring respective portions of the tubular tissue segments being connected to one another. For example, the head assembly 114 may include a circular knife 136. The circular knife 136 may be movably positioned within the central bore 124 of the outer shell 122. For example, the circular knife 136 may be configured to translate relative to the outer shell 122 along the longitudinal axis of the head assembly 114 between a retracted position and an extended position. The circular knife 136 may be formed as a tubular member having a cutting edge along the distal end of the circular knife 136 and a central aperture extending through the circular knife 136. In some embodiments, the circular knife 136 may be fixedly attached to the staple pusher 130. In this manner, the circular knife 136 may move along with the staple pusher 130 between the retracted position and the extended position. When the circular knife 136 is in the extended position, the cutting edge thereof may extend distally beyond the staple guide 128 to engage and cut radially inner portions of the tubular tissue segments. It will be appreciated that the illustrated head assembly 114 is merely one example embodiment, and that other shapes and configurations of the head assembly 114 may be used with the surgical stapler 100.

The trocar 116 may be formed as an elongated member extending along the longitudinal axis of the head assembly 114. The trocar 116 movably positioned within the tubular shaft 110 and/or the head assembly 114. For example, the trocar 116 may be configured to translate relative to the outer shell 122 along the longitudinal axis of the head assembly 114 between a retracted position, as shown in FIG. 1B, and an extended position, as shown in FIGS. 1A and 1C. When the trocar 116 is in the retracted position, a proximal portion of the trocar 116 may be positioned within the tubular shaft 110, and a distal portion of the trocar 116 may be positioned within the head assembly 114. When the trocar 116 is in the extended position, a proximal portion of the trocar 116 may be positioned within the head assembly 114, and a distal portion of the trocar 116 may extend distally beyond the distal end of the head assembly 114. As shown, the trocar 116 may include a distal tip 138 configured to penetrate tissue during use of the surgical stapler 100, as described below. The trocar 116 also may include a protrusion 140 extending along the outer circumference of an intermediate portion of the trocar 116 and configured to facilitate attachment of the anvil 118 to the trocar 116 during use of the surgical stapler 100. It will be appreciated that the illustrated trocar 116 is merely one example embodiment, and that other shapes and configurations of the trocar 116 may be used with the surgical stapler 100.

The anvil 118 may be formed as an elongated assembly configured for attachment to the trocar 116. As shown, the anvil 118 may include an anvil shaft 142 and an anvil head 144 attached to one another. In some embodiments, the anvil head 144 may be fixedly attached to the anvil shaft 142, with the anvil head 144 extending perpendicular to the longitudinal axis of the anvil shaft 142. In other embodiments, the anvil head 144 may be pivotably attached to the anvil shaft 142, such that the orientation of the anvil head 144 relative to the longitudinal axis of the anvil shaft 142 may be adjusted, for example, to ease insertion of the anvil head 144 into or removal of the anvil head 144 from a tubular tissue segment. The anvil shaft 142 may include a central passage 146 for receiving a distal portion of the trocar 116 therein, as shown in FIG. 1D. The anvil shaft 142 also may include a groove 148 extending along the inner circumference of the central passage 146 and configured to receive the protrusion 140 of the trocar 116 therein as well as one or more elongated slots 150 to facilitate a snap-fit connection between the anvil shaft 142 and the trocar 116. During use of the surgical stapler 100, the anvil 118 may be attached to the trocar 116 and then moved relative to the head assembly 114 from an extended position, as shown in FIG. 1D, to a retracted position, as shown in FIG. 1E. In this manner, the movement of the trocar 116 from its extended position to its retracted position may move the anvil 118 from its extended position to its retracted position. When the anvil 118 is in the retracted position, the anvil head 144 may be positioned adjacent to or near the distal end of the head assembly 114. In this manner, the anvil head 144 may facilitate desired deformation of the staples when the staples are ejected from the staple guide 128 and the free ends of the staples engage the anvil head 144. Additionally, the anvil head 144 may facilitate coring of the radially inner portions of the tissue segments when the circular knife 136 passes through the tissue segments and engages the anvil head 144. It will be appreciated that the illustrated anvil 118 is merely one example embodiment, and that other shapes and configurations of the anvil 118 may be used with the surgical stapler 100.

The surgical stapler 100 may include one or more components configured to allow the clinician to control movement of the trocar 116 and the anvil 118 relative to the head assembly 114 from outside of the patient during use of the stapler 100. For example, the surgical stapler 100 may include a knob 152 that is attached to the handle 112 and coupled to the trocar 116. The knob 152 may be rotatably attached to the handle 112 such that the knob 152 is configured to rotate about the longitudinal axis of the handle 112. The knob 152 may be mechanically coupled to the trocar 116 such that rotation of the knob 152 in a first direction (e.g., counter-clockwise) causes the trocar 116 to move distally relative to the head assembly 114 and rotation of the knob 152 in an opposite second direction (e.g., clockwise) causes the trocar 116 to move proximally relative to the head assembly 114. Various types of components may be used to mechanically couple the knob 152 to the trocar 116 in this manner, such as one or more threaded members, geared members, cams, couplings, and/or other mechanical components. It will be appreciated that such components may be positioned within respective portions of the tubular shaft 110, the handle 112, and/or the head assembly 114.

The surgical stapler 100 also may include one or more components configured to allow the clinician to control movement of the staple pusher 130 and the circular knife 136 relative to the outer shell 122 and the staple guide 128 from outside of the patient during use of the stapler 100. For example, the surgical stapler 100 may include a lever 154 that is attached to the handle 112 and coupled to the staple pusher 130. The lever 154 may be pivotably attached to the handle 112 such that the lever 154 is configured to pivot relative to the handle 112. The lever 154 may be mechanically coupled to the staple pusher 130 such that pivotal movement of the lever 154 in a first direction (e.g., toward the handle 112) causes the staple pusher 130 to move distally relative to the outer shell 122 and the staple guide 128 and pivotal movement of the lever 154 in an opposite second direction (e.g., away from the handle 112) causes the staple pusher 130 to move proximally relative to the outer shell 122 and the staple guide 128. Various types of components may be used to mechanically couple the lever 154 to the staple pusher 130 in this manner, such as one or more threaded members, geared members, cams, couplings, and/or other mechanical components. It will be appreciated that such components may be positioned within respective portions of the tubular shaft 110, the handle 112, and/or the head assembly 114.

The insertion guide 120 may be formed as an elongated assembly configured for engaging the head assembly 114. As shown, the insertion guide 120 may include an expandable member 162, a tube 164, a port 166, and a distal tip 168. The expandable member 162 may be configured to move or be transitioned between a collapsed configuration, as shown in FIG. 1F, and an expanded configuration, as shown in FIGS. 1A and 1G. For example, the expandable member 162 may be configured to radially expand and collapse about the longitudinal axis of the expandable member 162 between the collapsed configuration and the expanded configuration. In some embodiments, as shown, the expandable member 162 may be an inflatable atraumatic balloon that is formed of a flexible, elastomeric material, e.g., silicone, polyurethane, etc. In this manner, the expandable member 162 may include an internal reservoir 170 that is configured to receive a fluid, such as air, water, or saline, to facilitate expansion of the expandable member 162 from the collapsed configuration to the expanded configuration. In some embodiments, the expandable member 162 expands by elastic deformation upon being filled with the fluid. As shown, the expandable member 162 may have a curved outer surface when the expandable member 162 is in the expanded configuration. In this manner, when the expandable member 162 is in the expanded configuration, the curved outer surface may facilitate insertion of the expandable member 162 into the patient and advancement of the expandable member 162 through surrounding anatomy and to a target location within the patient, without causing injury to the patient. According to various embodiments, the expandable member 162 may have a spherical shape, an egg shape (e.g., ovoidal), a toroidal shape, or other shape having a curved outer surface when the expandable member 162 is in the expanded configuration. It will be appreciated that the illustrated expandable member 162 is merely one example embodiment, and that other shapes and configurations of the expandable member 162 may be used with the surgical stapler 100.

The tube 164 of the insertion guide 120 may be formed as an elongated, hollow member extending from the proximal end of the expandable member 162. The tube 164 may be fixedly attached to the expandable member 162. In some embodiments, as shown, a distal portion of the tube 164 may be positioned within the expandable member 162. The tube 164 may include an internal lumen 172 that extends from the proximal end of the tube 164 toward the distal end of the tube 164 and is in fluid communication with the internal reservoir 170 of the expandable member 162. For example, the lumen 172 may terminate at an opening 174 that is defined in a sidewall of the tube 164 and provides fluid communication between the lumen 172 and the internal reservoir 170 of the expandable member 162. In this manner, a fluid may be delivered through the lumen 172 and into the internal reservoir 170 to facilitate expansion of the expandable member 162 from the collapsed configuration to the expanded configuration. Similarly, the fluid may be subsequently released from the internal reservoir 170 and through the lumen 172 to facilitate contraction of the expandable member 162 from the expanded configuration to the collapsed configuration. In some embodiments, the tube 164 may be formed of a flexible material configured to allow the shape of the tube 164 to be manipulated during use of the insertion guide 120. In other embodiments, the tube 164 may be formed of a rigid or substantially rigid material configured to maintain a predetermined shape of the tube 164 during use. It will be appreciated that the illustrated tube 164 is merely one example embodiment, and that other shapes and configurations of the tube 164 may be used with the surgical stapler 100.

The port 166 of the insertion guide 120 may be fixedly attached to the proximal end of the tube 164 and configured to facilitate attachment of a fluid delivery device, such as a syringe. In this manner, the fluid delivery device may be used to deliver the fluid through the tube 164 and into the expandable member 162 or to withdraw the fluid from the expandable member 162 and the tube 164. In some embodiments, the port 166 may include a luer lock connection or other type of connection for attaching the fluid delivery device thereto. It will be appreciated that the illustrated port 166 is merely one example embodiment, and that other shapes and configurations of the port 166 may be used with the surgical stapler 100.

The distal tip 168 of the insertion guide 120 may be formed as a non-expandable, elongated member extending from the distal end of the expandable member 162. The distal tip 168 may be fixedly attached to the expandable member 162. In some embodiments, as shown, a proximal portion of the distal tip 168 may be positioned within the expandable member 162. In some embodiments, the distal tip 168 may be hollow. In other embodiments, the distal tip 168 may be solid. In some embodiments, the distal tip 168 may be formed of a flexible material configured to allow the distal tip 168 to elastically deform as the distal tip 168 engages surrounding anatomy of the patient during use of the insertion guide 120. In other embodiments, the distal tip 168 may be formed of a rigid or substantially rigid material configured to maintain a predetermined shape of the distal tip 168 during use. It will be appreciated that the illustrated distal tip 168 is merely one example embodiment, and that other shapes and configurations of the distal tip 168 may be used with the surgical stapler 100. In some embodiments, the distal tip 168 may be omitted, such that the distal end of the expandable member 162 defines the distal end of the insertion guide 120.

In some embodiments, as shown in FIGS. 1F and 1G, the insertion guide 120 may be inserted through the head assembly 114 of the surgical stapler 100. In particular, the insertion guide 120 may be inserted through the head assembly 114 such that a distal portion of the insertion guide 120 extends distally beyond the distal end face 132 of the head assembly 114, a proximal portion of the insertion guide 120 extends proximally from the head assembly 114, and an intermediate portion of the insertion guide 120 is positioned within the head assembly 114. As shown in FIG. 1F, the insertion guide 120 may be advanced through the head assembly 114 while the expandable member 162 is in the collapsed configuration. Similarly, the insertion guide 120 may be removed from the head assembly 114 while the expandable member 162 is in the collapsed configuration. As described above, the insertion guide 120 may extend through the lateral opening 126 of the head assembly 114, through the central bore 124 of the head assembly 114, and distally beyond the distal end face 132 of the head assembly 114. In this manner, the insertion guide 120 also may extend through the central aperture of the staple guide 128, through the opening of the staple pusher 130, and through the central aperture of the circular knife 136. As shown, the head assembly 114 may have a first outer diameter OD1 and the expandable member 162 may have a second outer diameter OD2 when the expandable member 162 is in the collapsed configuration, with the second outer diameter OD2 being less than the first outer diameter OD1.

After inserting the insertion guide 120 through the head assembly 114, the expandable member 162 may be moved from the collapsed configuration to the expanded configuration, as shown in FIG. 1G. In some embodiments, the expandable member 162 may engage the distal end face 132 of the head assembly 114 when the expandable member 162 is in the expanded configuration. In some embodiments, a proximal portion of the expandable member 162 may be positioned within the central bore 124 of the head assembly 114 when the expandable member 162 is in the expanded configuration. In this manner, the proximal portion of the expandable member 162 may assist in self-centering the expandable member 162 relative to the longitudinal axis of the head assembly 114 when the expandable member 162 is moved from the collapsed configuration to the expanded configuration. In other embodiments, the entirety of the expandable member 162 may be positioned outside of the central bore 124 of the head assembly 114 and distally beyond the distal end face 132 when the expandable member 162 is in the expanded configuration. As shown, the expandable member 162 may have a third outer diameter OD3 when the expandable member 162 is in the expanded configuration. In some embodiments, the third outer diameter OD3 may be greater than the first outer diameter OD1 of the head assembly 114. In other embodiments, the third outer diameter OD3 may be equal to the first outer diameter OD1 of the head assembly 114.

In some embodiments, as shown in FIGS. 1H and 11, the insertion guide 120 may be inserted through the tubular shaft 110 and the head assembly 114 of the surgical stapler 100. In particular, the insertion guide 120 may be inserted through the tubular shaft 110 and the head assembly 114 such that a distal portion of the insertion guide 120 extends distally beyond the distal end face 132 of the head assembly 114, a proximal portion of the insertion guide 120 extends proximally from the tubular shaft 110, and an intermediate portion of the insertion guide 120 is positioned within the tubular shaft 110 and the head assembly 114. In some embodiments, the insertion guide 120 may extend through the lumen of the tubular shaft 110 along with other internal components of the surgical stapler 100. In some embodiments, the tubular shaft 110 may include a dedicated tube, passage, or channel positioned within the lumen of the tubular shaft 110 and configured to receive the insertion guide 120 therethrough. In this manner, the tubular shaft 110 may have a predefined pathway for allowing the insertion guide 120 to extend therethrough and to shield the insertion guide 120 from other internal components within the tubular shaft 110. As shown in FIG. 1H, the insertion guide 120 may be advanced through the tubular shaft 110 and the head assembly 114 while the expandable member 162 is in the collapsed configuration. Similarly, the insertion guide 120 may be removed from the tubular shaft 110 and the head assembly 114 while the expandable member 162 is in the collapsed configuration. In some embodiments, the insertion guide 120 may extend through a lateral opening 126′ of the handle 112, through a portion of the internal space of the handle 112, through the lumen of the tubular shaft 110, through the central bore 124 of the head assembly 114, and distally beyond the distal end face 132 of the head assembly 114. In this manner, the insertion guide 120 also may extend through the central aperture of the staple guide 128, through an opening of the staple pusher 130, and through the central aperture of the circular knife 136. As shown, the head assembly 114 may have a first outer diameter OD1 and the expandable member 162 may have a second outer diameter OD2 when the expandable member 162 is in the collapsed configuration, with the second outer diameter OD2 being less than the first outer diameter OD1.

After inserting the insertion guide 120 through the tubular shaft 110 and the head assembly 114, the expandable member 162 may be moved from the collapsed configuration to the expanded configuration, as shown in FIG. 1I. In some embodiments, the expandable member 162 may engage the distal end face 132 of the head assembly 114 when the expandable member 162 is in the expanded configuration. In some embodiments, a proximal portion of the expandable member 162 may be positioned within the central bore 124 of the head assembly 114 when the expandable member 162 is in the expanded configuration. In this manner, the proximal portion of the expandable member 162 may assist in self-centering the expandable member 162 relative to the longitudinal axis of the head assembly 114 when the expandable member 162 is moved from the collapsed configuration to the expanded configuration. In other embodiments, the entirety of the expandable member 162 may be positioned outside of the central bore 124 of the head assembly 114 and distally beyond the distal end face 132 when the expandable member 162 is in the expanded configuration. As shown, the expandable member 162 may have a third outer diameter OD3 when the expandable member 162 is in the expanded configuration. In some embodiments, the third outer diameter OD3 may be greater than the first outer diameter OD1 of the head assembly 114. In other embodiments, the third outer diameter OD3 may be equal to the first outer diameter OD1 of the head assembly 114.

In some embodiments, the surgical stapler 100 may include both the lateral opening 126 of the head assembly 114 and the lateral opening 126′ of the handle 112, as shown in FIG. 1A. In this manner, the clinician may have the option of using the lateral opening 126 of the head assembly 114 to extend the insertion guide 120 through only the head assembly 114, as described above with respect to FIGS. 1F and 1G, or using the lateral opening 126′ of the handle 112 to extend the insertion guide 120 through both the tubular shaft 110 and the head assembly 114, as described above with respect to FIGS. 1H and 11. In other embodiments, the lateral opening 126 of the head assembly 114 or the lateral opening 126′ of the handle 112 may be omitted.

Methods of Use

FIGS. 2A-2H illustrate an example method of using the surgical stapler 100 to perform an end-to-end anastomosis between two tissue segments of a tubular tissue structure of a patient, in accordance with one or more embodiments of the disclosure. FIG. 2A shows a tubular tissue structure 200 of a patient, which includes a first tissue segment 202, a second tissue segment 204, and an intermediate portion 206 of the tubular tissue structure 200 to be removed from the patient. In some embodiments, the tubular tissue structure 200 may be a colon of the patient, the first tissue segment 202 may be an upper colon segment, and the second tissue segment 204 may be a lower colon segment. Following resection of the intermediate portion 206, the first tissue segment 202 and the second tissue segment 204 may need to be reconnected to one another by performing an end-to-end anastomosis to restore the natural function of the tubular tissue structure 200.

The anastomosis procedure may begin by inserting the anvil head 144 of the anvil 118 into the first tissue segment 202 and securing the free end of the first tissue segment 202 around the anvil shaft 142 of the anvil 118, as shown in FIG. 2B. In this manner, the anvil shaft 142 may extend outside of the first tissue segment 202, while the anvil head 144 is maintained within the first tissue segment 202. In some embodiments, the free end of the first tissue segment 202 may be secured around the anvil shaft 142 by suturing the tissue, for example, to form a purse-string suture 208. Meanwhile, the free end of the second tissue segment 204 may be closed by suturing the tissue, for example, to form a linear suture 210, as shown in FIG. 2B.

After closing the free end of the second tissue segment 204, the head assembly 114 of the surgical stapler 100 may be inserted into the patient and advanced toward the closed end of the second tissue segment 204. As described above, the insertion guide 120 may be used to facilitate insertion of the head assembly 114 and advancement of the head assembly 114 to the closed end of the second tissue segment 204. In particular, the insertion guide 120 may be inserted through the head assembly 114, as described above with respect to FIG. 1F, and the expandable member 162 may be moved from the collapsed configuration to the expanded configuration, as described above with respect to FIG. 1G. Alternatively, the insertion guide 120 may be inserted through the tubular shaft 110 and the head assembly 114, as described above with respect to FIG. 1H, and the expandable member 162 may be moved from the collapsed configuration to the expanded configuration, as described above with respect to FIG. 1I. For example, the expandable member 162 may be expanded to the expanded configuration by delivering a fluid through the tube 164 and into the internal reservoir 170 of the expandable member 162 via a fluid delivery device attached to the port 166. The insertion guide 120 and the head assembly 114 then may be inserted into the patient and advanced through the surrounding anatomy until the expandable member 162 and the head assembly 114 are positioned adjacent to the closed end of the second tissue segment 204, as shown in FIG. 2C. Meanwhile, the port 166 and the fluid delivery device may remain positioned outside of the patient. In embodiments in which the tubular tissue structure 200 is the colon of the patient and the second tissue segment 204 is the lower colon segment, the expandable member 162 may ease insertion of the head assembly 114 through the anus, through the contours of the rectum, and through the lower colon segment, while inhibiting the distal end face 132 of the head assembly 114 from engaging the surrounding anatomy.

After the expandable member 162 and the head assembly 114 are positioned adjacent to the closed end of the second tissue segment 204, the expandable member 162 may be moved from the expanded configuration to the collapsed configuration. For example, the fluid delivery device may be used to withdraw the fluid from the internal reservoir 170 of the expandable member 162, thereby causing the expandable member 162 to assume the collapsed configuration. The insertion guide 120 then may be withdrawn proximally from the head assembly 114 and removed from the patient. After removal of the insertion guide 120 from the head assembly 114, the head assembly 114 may be further advanced to a position adjacent to the closed end of the second tissue segment 204, as shown in FIG. 2D.

With the head assembly 114 positioned adjacent to the closed end of the second tissue segment 204, the trocar 116 may be passed through the closed end of the second tissue segment 204, as shown in FIG. 2E. For example, the knob 152 of the surgical stapler 100 may be rotated counter-clockwise to cause the trocar 116 to move from the retracted position to the extended position. In this manner, the trocar 116 may be advanced through the closed end of the second tissue segment 204 and extend into the space between the first tissue segment 202 and the second tissue segment 204.

The anvil 118 and the trocar 116 then may be attached to one another, as shown in FIG. 2F. For example, the anvil 118 and/or the trocar 116 may be grasped via one or more surgical instruments, such as forceps, and moved relative to one another such that the anvil 118 is advanced over the trocar 116 and secured thereto by the connection between the protrusion 140 and the groove 148.

After attaching the anvil 118 to the trocar 116, the anvil 118 and the trocar 116 may be moved from the extended position to the retracted position, as shown in FIG. 2G. For example, the knob 152 of the surgical stapler 100 may be rotated clockwise to cause the trocar 116 and the anvil 118 to move from the extended position to the retracted position. In this manner, the respective ends of the first tissue segment 202 and the second tissue segment 204 may be drawn together and captured between the anvil head 144 and the distal end face 132 of the head assembly 114. The head assembly 114 then may be actuated to facilitate formation of a circular array of staples through the respective end portions of the first tissue segment 202 and the second tissue segment 204 and to core respective inner portions of the first tissue segment 202 and the second tissue segment 204. For example, the lever 154 of the surgical stapler 100 may be moved relative to the handle 112 to actuate the head assembly 114. The actuation of the head assembly 114 may cause the staple pusher 130 and the circular knife 136 to move distally relative to the outer shell 122 and the staple guide 128. As described above, the movement of the staple pusher 130 may cause the staples to be ejected from the staple guide 128, advanced through the respective end portions of the first tissue segment 202 and the second tissue segment 204, and deformed against the anvil head 144. In this manner, a circular array of staples 212 may be formed to reconnect the first tissue segment 202 and the second tissue segment 204. Meanwhile, the movement of the circular knife 136 may cause the cutting edge thereof to cut the respective inner portions of the first tissue segment 202 and the second tissue segment 204 against the anvil head 144. In this manner, fluid communication between the first tissue segment 202 and the second tissue segment 204 may be restored. After actuating the head assembly 114, the surgical stapler 100 may be removed from the patient, leaving the first tissue segment 202 and the second tissue segment 204 reconnected by an end-to-end anastomosis, as shown in FIG. 2H.

Insertion Guides

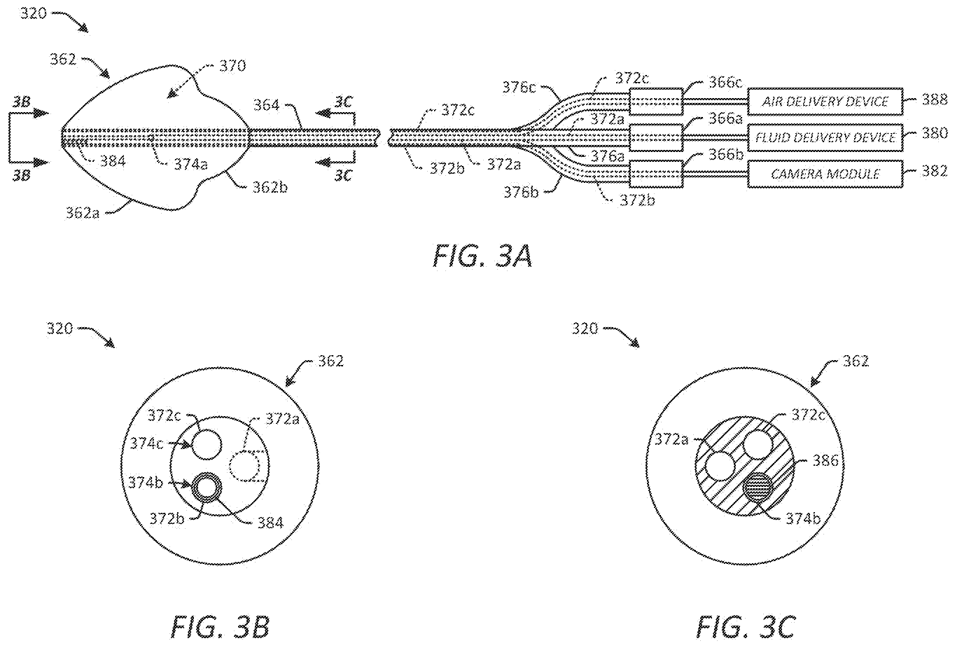

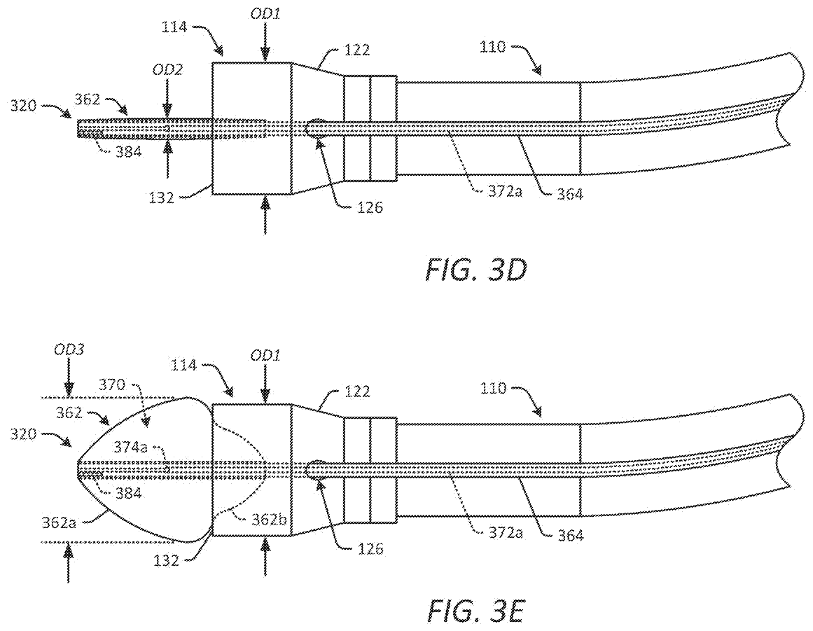

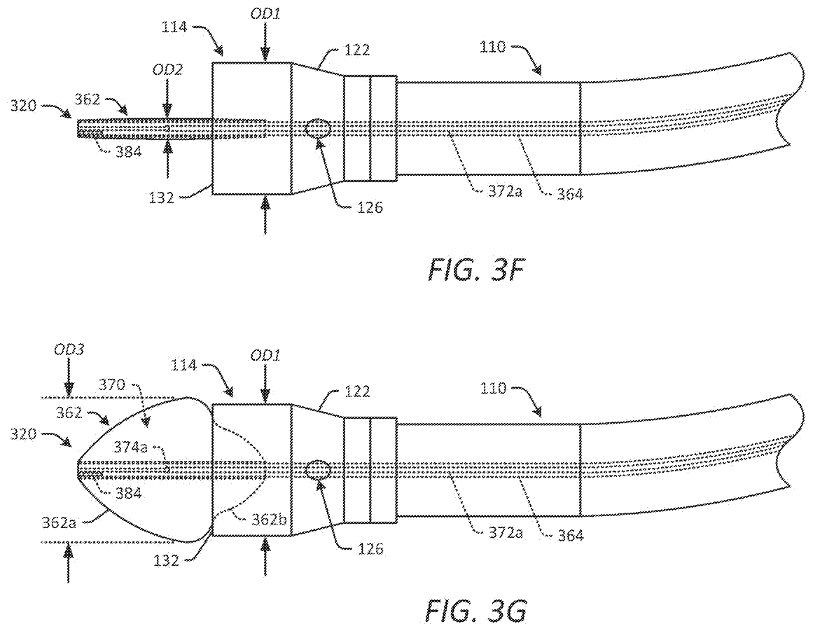

FIGS. 3A-3G illustrate an insertion guide 320 as may be used with the surgical stapler 100 in accordance with one or more embodiments of the disclosure. In some embodiments, the insertion guide 320 may be used with the surgical stapler 100 instead of the insertion guide 120 described above. Similar to the insertion guide 120, the insertion guide 320 may be used to facilitate insertion of the head assembly 114 into the patient and to guide advancement of the head assembly 114 to a target location for performing an anastomosis. Once the head assembly 114 is positioned at or near the target location, the insertion guide 320 may be removed from stapler and from the patient, and the head assembly 114, the trocar 116, and the anvil 118 then may be used in a conventional manner to connect two tubular tissue segments via a circular array of staples. As described below, the insertion guide 320 may have a multi-lumen configuration that facilitates visualization of the internal anatomy of a patient as well as air insufflation of a body cavity of the patient. Accordingly, the insertion guide 320 may be used with the surgical stapler 100 in instances in which a clinician desires to view the internal anatomy and/or expand the body cavity as the head assembly 114 is advanced to a target location within the patient. For example, when the surgical stapler 100 is used to perform an end-to-end anastomosis between an upper colon segment and a lower colon segment, the insertion guide 320 may be used to facilitate visualization and expansion of the rectum and the lower colon segment of the patient.

As shown in FIG. 3A, the insertion guide 320 may be formed as an elongated assembly configured for engaging the head assembly 114 of the surgical stapler 100. The insertion guide 320 may include an expandable member 362, a tube 364, and a plurality of ports 366a, 366b, 366c. The expandable member 362 may be configured to move or be transitioned between a collapsed configuration, as shown in FIG. 3D, and an expanded configuration, as shown in FIGS. 3A and 3E. For example, the expandable member 362 may be configured to radially expand and collapse about the longitudinal axis of the expandable member 362 between the collapsed configuration and the expanded configuration. In some embodiments, as shown, the expandable member 362 may be an inflatable atraumatic balloon that is formed of a flexible, elastomeric material, e.g., silicone, polyurethane, etc. In this manner, the expandable member 362 may include an internal reservoir 370 that is configured to receive a fluid, such as air, water, or saline, to facilitate expansion of the expandable member 362 from the collapsed configuration to the expanded configuration. In some embodiments, the expandable member 362 expands by elastic deformation upon being filled with the fluid. As shown, the expandable member 362 may have a curved outer surface when the expandable member 362 is in the expanded configuration. In this manner, when the expandable member 362 is in the expanded configuration, the curved outer surface may facilitate insertion of the expandable member 362 into the patient and advancement of the expandable member 362 through surrounding anatomy and to a target location within the patient, without causing injury to the patient. According to various embodiments, the expandable member 362 may have an egg shape (i.e., an ovoid), a spherical shape, a toroidal shape, or other shape having a curved outer surface when the expandable member 362 is in the expanded configuration. As shown, the expandable member 362 may include a distal portion 362a having a partial egg shape and a proximal portion 362b having a partial egg shape. In some embodiments, the distal portion 362a may have a maximum outer diameter that is greater than a maximum outer diameter of the proximal portion 362b. In some embodiments, the distal portion 362a may have a length that is greater than a length of the proximal portion 362b. As described below, during use of the insertion guide 320, the distal portion 362a may be configured to be positioned outside of the head assembly 114, and the proximal portion 362b may be configured to be positioned at least partially within the head assembly 114. It will be appreciated that the illustrated expandable member 362 is merely one example embodiment, and that other shapes and configurations of the expandable member 362 may be used with the surgical stapler 100.

The tube 364 of the insertion guide 320 may be formed as an elongated, hollow member extending from the proximal end of the expandable member 362. The tube 364 may be fixedly attached to the expandable member 362. In some embodiments, as shown, a distal portion of the tube 364 may be positioned within the expandable member 362. The tube 364 may include a plurality of internal lumens 372a, 372b, 372c defined therein and extending along the length of the tube 364. In some embodiments, as shown, the tube 364 may include a plurality of tubular branches 376a, 376b, 376c positioned at the proximal end of the tube 364 and attached to the respective ports 366a, 366b, 366c. In particular, the first port 366a may be attached to the first branch 376a, the second port 366b may be attached to the second branch 376b, and the third port 366c may be attached to the third branch 376c. As shown, the first lumen 372a may extend from the proximal end of the first branch 376a toward the distal end of the tube 364 and be in fluid communication with the internal reservoir 370 of the expandable member 362. For example, the first lumen 372a may terminate at a first opening 374a that is defined in a sidewall of the tube 364 and provides fluid communication between the first lumen 372a and the internal reservoir 370 of the expandable member 362. In this manner, a fluid may be delivered through the first lumen 372a and into the internal reservoir 370 to facilitate expansion of the expandable member 362 from the collapsed configuration to the expanded configuration. Similarly, the fluid may be subsequently released from the internal reservoir 370 and through the first lumen 372a to facilitate contraction of the expandable member 362 from the expanded configuration to the collapsed configuration. As shown, the second lumen 372b may extend from the proximal end of the second branch 376b to the distal end of the tube 364. For example, the second lumen 372b may terminate at a second opening 374b defined in the distal end of the tube 364. As described below, the second lumen 372b may be configured to receive a camera therein to facilitate visualization of the internal anatomy of a patient during use of the insertion guide 320. As shown, the third lumen 372c may extend from the proximal end of the third branch 376c to the distal end of the tube 364. For example, the third lumen 372c may terminate at a third opening 374c defined in the distal end of the tube 364. As described below, the third lumen 372c may be configured to allow air to be delivered therethrough to facilitate air insufflation of a body cavity of a patient during use of the insertion guide 320. In some embodiments, the tube 364 may be formed of a flexible material configured to allow the shape of the tube 364 to be manipulated during use of the insertion guide 320. In other embodiments, the tube 364 may be formed of a rigid or substantially rigid material configured to maintain a predetermined shape of the tube 364 during use. It will be appreciated that the illustrated tube 364 is merely one example embodiment, and that other shapes and configurations of the tube 364 may be used with the surgical stapler 100.

The first port 366a of the insertion guide 320 may be fixedly attached to the proximal end of the first branch 376a and configured to facilitate attachment of a fluid delivery device 380 thereto. In this manner, the fluid delivery device 380 may be used to deliver the fluid through the first lumen 372a of the tube 364 and into the expandable member 362 or to withdraw the fluid from the expandable member 362 and the first lumen 372a of the tube 364. In some embodiments, the first port 366a may include a luer lock connection or other type of connection for attaching the fluid delivery device 380 thereto. In some embodiments, the fluid delivery device 380 may be a syringe, although other types of devices for delivering and withdrawing the fluid may be used. The second port 366b may be fixedly attached to the proximal end of the second branch 376b and configured to facilitate attachment of a camera module 382 thereto. The camera module 382 may be in operable communication with a camera 384 that is configured to be positioned at least partially within the second lumen 372b of the tube 364. In some embodiments, as shown, a cable 386, such as a fiber optic cable, may extend between the camera module 382 and the camera 384 and be configured to be positioned within the second lumen 372b of the tube 364. In this manner, the camera 384 may be inserted through the second lumen 372b via the cable 386. In some embodiments, the second port 366b may include a luer lock connection or other type of connection for attaching the camera module 382 thereto. The third port 366c may be fixedly attached to the proximal end of the third branch 376c and configured to facilitate attachment of an air delivery device 388 thereto. In this manner, the air delivery device 388 may be used to deliver air through the third lumen 372c of the tube 364 for air insufflation of a body cavity of a patient. In some embodiments, the third port 366c may include a luer lock connection or other type of connection for attaching the air delivery device 388 thereto. In some embodiments, the air delivery device 388 may be a pump, although other types of devices for delivering the air may be used. It will be appreciated that the illustrated ports 366a, 366b, 366c are merely one example embodiment, and that other shapes and configurations of the ports 366a, 366b, 366c may be used with the surgical stapler 100.

As shown, the camera 384 may be positioned within the second lumen 372b of the tube 364 and at least partially within the expandable member 362 of the insertion guide 320. In some embodiments, the camera 384 may be removably positioned within the second lumen 372b. For example, the camera 384 and the cable 386 may be inserted into the second lumen 372b prior to use of the insertion guide 320. In some embodiments, the camera 384 may be fixedly attached to the tube 364 and/or the expandable member 362. For example, the camera 384 and/or the cable 386 may be fixedly secured within the second lumen 372b. In some embodiments, the camera 384 may be positioned entirely within the tube 364 and the expandable member 362. In some embodiments, a distal end of the camera 384 may be flush with the distal end of the tube 364 and/or the distal end of the expandable member 362. In some embodiments, the camera 384 may be positioned at least partially outside of the tube 364 and the expandable member 362 such that a distal end portion of the camera 384 extends distally beyond the distal end of the tube 364 and/or the distal end of the expandable member 362. During use, the camera 384 may be configured to capture video data, image data, or both, of the patient's internal anatomy positioned distally beyond the expandable member 362. The video and/or image data may be transmitted from the camera 384 to the camera module 382, for example, via the cable 386. The camera module 382 may include a display screen or may be connected to a display screen configured to display the video and/or image data. In this manner, the clinician may view the video and/or image data in real time to analyze the internal anatomy of the patient. Such visualization of the patient's anatomy may assist the clinician in advancing the insertion guide 320 and the head assembly 114 of the surgical stapler 100 to a target location within the patient. In some embodiments, the clinician may insufflate the body cavity of the patient by delivering air through the third lumen 372c and into the body cavity. Such air insufflation may ease advancement of the insertion guide 320 and the head assembly 114 of the surgical stapler 100 to the target location and also may improve visualization of the internal anatomy via the camera 384.

Although the illustrated embodiment of the insertion guide 320 includes three lumens 372a, 372b, 372c and three ports 366a, 366b, 366c, the insertion guide 320 may include any number of lumens and any number of ports in other embodiments. In some embodiments, one of the lumens 372a, 372b, 372c may be used for multiple alternative purposes. For example, the second lumen 372b may be used for receiving the camera 384 therein when the clinician desires to visualize the internal anatomy of the patient or, alternatively, the second lumen 372b may be used to deliver air through the insertion guide 320 when the clinician desires to insufflate the body cavity of the patient with air. In such embodiments, the third lumen 372c and the third port 366c may be omitted, and either the camera module 382 or the air delivery device 388 may be attached to the second port 366b, depending on how the clinician desires to utilize the second lumen 372b. Further, it will be appreciated that various shapes, sizes, and configurations of the lumens 372a, 372b, 372c of the insertion guide 320 may be used. For example, although the illustrated embodiment shows the lumens 372a, 372b, 372c as having circular cross-sectional shapes, other shapes, such as oval, square, rectangular, triangular, or irregular cross-sectional shapes may be used in other embodiments. Additionally, although the illustrated embodiment shows the lumens 372a, 372b, 372c as having similar sizes and being arranged in a circular array about the longitudinal axis of the tube 364, the lumens 372a, 372b, 372c may have different sizes from one another and may be arranged in other configurations relative to one another in other embodiments.

In some embodiments, as shown in FIGS. 3D and 3E, the insertion guide 320 may be inserted through the head assembly 114 of the surgical stapler 100. In particular, the insertion guide 320 may be inserted through the head assembly 114 such that a distal portion of the insertion guide 320 extends distally beyond the distal end face 132 of the head assembly 114, a proximal portion of the insertion guide 320 extends proximally from the head assembly 114, and an intermediate portion of the insertion guide 320 is positioned within the head assembly 114. As shown in FIG. 3D, the insertion guide 320 may be advanced through the head assembly 114 while the expandable member 362 is in the collapsed configuration. Similarly, the insertion guide 320 may be removed from the head assembly 114 while the expandable member 362 is in the collapsed configuration. As described above, the insertion guide 320 may extend through the lateral opening 326 of the head assembly 114, through the central bore 124 of the head assembly 114, and distally beyond the distal end face 132 of the head assembly 114. In this manner, the insertion guide 320 also may extend through the central aperture of the staple guide 128, through the opening of the staple pusher 130, and through the central aperture of the circular knife 136. As shown, the head assembly 114 may have a first outer diameter OD1 and the expandable member 362 may have a second outer diameter OD2 when the expandable member 362 is in the collapsed configuration, with the second outer diameter OD2 being less than the first outer diameter OD1.

After inserting the insertion guide 320 through the head assembly 114, the expandable member 362 may be moved from the collapsed configuration to the expanded configuration, as shown in FIG. 3E. In some embodiments, the expandable member 362 may engage the distal end face 132 of the head assembly 114 when the expandable member 362 is in the expanded configuration. In some embodiments, the distal portion 362a of the expandable member 362 may be positioned outside of the head assembly 114 and the proximal portion 362b of the expandable member 362 may be positioned within the central bore 124 of the head assembly 114 when the expandable member 362 is in the expanded configuration. In this manner, the proximal portion 362b of the expandable member 362 may assist in self-centering the expandable member 362 relative to the longitudinal axis of the head assembly 114 when the expandable member 362 is moved from the collapsed configuration to the expanded configuration. In other embodiments, the entirety of the expandable member 362 may be positioned outside of the central bore 124 of the head assembly 114 and distally beyond the distal end face 132 when the expandable member 362 is in the expanded configuration. As shown, the expandable member 362 may have a third outer diameter OD3 when the expandable member 362 is in the expanded configuration. In some embodiments, the third outer diameter OD3 may be greater than the first outer diameter OD1 of the head assembly 114. In other embodiments, the third outer diameter OD3 may be equal to the first outer diameter OD1 of the head assembly 114.

In some embodiments, as shown in FIGS. 3F and 3G, the insertion guide 320 may be inserted through the tubular shaft 110 and the head assembly 114 of the surgical stapler 100. In particular, the insertion guide 320 may be inserted through the tubular shaft 110 and the head assembly 114 such that a distal portion of the insertion guide 320 extends distally beyond the distal end face 132 of the head assembly 114, a proximal portion of the insertion guide 320 extends proximally from the tubular shaft 110, and an intermediate portion of the insertion guide 320 is positioned within the tubular shaft 110 and the head assembly 114. In some embodiments, the insertion guide 320 may extend through the lumen of the tubular shaft 110 along with other internal components of the surgical stapler 100. In some embodiments, the tubular shaft 110 may include a dedicated tube, passage, or channel positioned within the lumen of the tubular shaft 110 and configured to receive the insertion guide 320 therethrough. In this manner, the tubular shaft 110 may have a predefined pathway for allowing the insertion guide 320 to extend therethrough and to shield the insertion guide 320 from other internal components within the tubular shaft 110. As shown in FIG. 3F, the insertion guide 320 may be advanced through the tubular shaft 110 and the head assembly 114 while the expandable member 362 is in the collapsed configuration. Similarly, the insertion guide 320 may be removed from the tubular shaft 110 and the head assembly 114 while the expandable member 362 is in the collapsed configuration. In some embodiments, the insertion guide 320 may extend through the lateral opening 126′ of the handle 112, through a portion of the internal space of the handle 112, through the lumen of the tubular shaft 110, through the central bore 124 of the head assembly 114, and distally beyond the distal end face 132 of the head assembly 114. In this manner, the insertion guide 120 also may extend through the central aperture of the staple guide 128, through an opening of the staple pusher 130, and through the central aperture of the circular knife 136. As shown, the head assembly 114 may have a first outer diameter OD1 and the expandable member 362 may have a second outer diameter OD2 when the expandable member 362 is in the collapsed configuration, with the second outer diameter OD2 being less than the first outer diameter OD1.

After inserting the insertion guide 320 through the tubular shaft 110 and the head assembly 114, the expandable member 362 may be moved from the collapsed configuration to the expanded configuration, as shown in FIG. 3G. In some embodiments, the expandable member 362 may engage the distal end face 132 of the head assembly 114 when the expandable member 362 is in the expanded configuration. In some embodiments, the distal portion 362a of the expandable member 362 may be positioned outside of the head assembly 114 and the proximal portion 362b of the expandable member 362 may be positioned within the central bore 124 of the head assembly 114 when the expandable member 362 is in the expanded configuration. In this manner, the proximal portion 362b of the expandable member 362 may assist in self-centering the expandable member 362 relative to the longitudinal axis of the head assembly 114 when the expandable member 362 is moved from the collapsed configuration to the expanded configuration. In other embodiments, the entirety of the expandable member 362 may be positioned outside of the central bore 124 of the head assembly 114 and distally beyond the distal end face 132 when the expandable member 362 is in the expanded configuration. As shown, the expandable member 362 may have a third outer diameter OD3 when the expandable member 362 is in the expanded configuration. In some embodiments, the third outer diameter OD3 may be greater than the first outer diameter OD1 of the head assembly 114. In other embodiments, the third outer diameter OD3 may be equal to the first outer diameter OD1 of the head assembly 114.

The insertion guide 320 may be used with the surgical stapler 100 in a manner similar to the insertion guide 120 described above with respect to FIGS. 2A-2H in performing an end-to-end anastomosis between two tissue segments of a tubular tissue structure of a patient. As described above, after closing the free end of the second tissue segment 204, the head assembly 114 of the surgical stapler 100 may be inserted into the patient and advanced toward the closed end of the second tissue segment 204. The insertion guide 320 may be used to facilitate insertion of the head assembly 114 and advancement of the head assembly 114 to the closed end of the second tissue segment 204. In particular, the insertion guide 320 may be inserted through the head assembly 114, as described above with respect to FIG. 3D, and the expandable member 362 may be moved from the collapsed configuration to the expanded configuration, as described above with respect to FIG. 3E. Alternatively, the insertion guide 320 may be inserted through the tubular shaft 110 and the head assembly 114, as described above with respect to FIG. 3F, and the expandable member 362 may be moved from the collapsed configuration to the expanded configuration, as described above with respect to FIG. 3G. For example, the expandable member 362 may be expanded to the expanded configuration by delivering a fluid through the first lumen 372a of the tube 364 and into the internal reservoir 370 of the expandable member 362 via the fluid delivery device 380 attached to the first port 366a. The insertion guide 320 and the head assembly 114 then may be inserted into the patient. After initial insertion of the expandable member 362, the body cavity of the patient may insufflated with air, if desired. For example, air may be delivered from the air delivery device 388 through the third lumen 372c of the tube 364 and into the patient's body cavity to facilitate expansion of the body cavity. The insertion guide 320 and the head assembly 114 then may be advanced through the surrounding anatomy until the expandable member 362 and the head assembly 114 are positioned adjacent to the closed end of the second tissue segment 204. Meanwhile, the ports 366a, 366b, 366c, the fluid delivery device 380, the camera module 382, and the air delivery device 388 may remain positioned outside of the patient. As the insertion guide 320 and the head assembly 114 are advanced through the patient to the closed end of the second tissue segment 204, the camera 384 and the camera module 382 may be used to visualize the internal anatomy of the patient. For example, the clinician may view real-time video and/or image data that is captured by the camera 384 and displayed at a display screen of the camera module 382 or connected thereto. In this manner, air insufflation of the body cavity and/or visualization of the internal anatomy of the patient may allow the clinician to more easily advance the expandable member 362 and the head assembly 114 to the closed end of the second tissue segment 204. In embodiments in which the tubular tissue structure 200 is the colon of the patient and the second tissue segment 204 is the lower colon segment, the expandable member 362 may ease insertion of the head assembly 114 through the anus, through the contours of the rectum, and through the lower colon segment, while inhibiting the distal end face 132 of the head assembly 114 from engaging the surrounding anatomy.

After the expandable member 362 and the head assembly 114 are positioned adjacent to the closed end of the second tissue segment 204, the expandable member 362 may be moved from the expanded configuration to the collapsed configuration. For example, the fluid delivery device 380 may be used to withdraw the fluid from the internal reservoir 370 of the expandable member 362, thereby causing the expandable member 362 to assume the collapsed configuration. The insertion guide 320 then may be withdrawn proximally from the head assembly 114 and removed from the patient. After removal of the insertion guide 320 from the head assembly 114, the head assembly 114 may be further advanced to a position adjacent to the closed end of the second tissue segment 204. The remaining steps of the method for performing an end-to-end anastomosis between two tissue segments of a tubular tissue structure of a patient may be carried out in the same manner described above with respect to FIGS. 2A-2H.

### Exemplary Embodiments

Embodiment 1. A surgical stapler comprising: a tubular shaft having a proximal end and a distal end; a handle extending from the proximal end of the tubular shaft; a head assembly extending from the distal end of the tubular shaft; and an insertion guide configured to extend through the head assembly, the insertion guide comprising: (i) an expandable member configured to move between a collapsed configuration and an expanded configuration; and (ii) a camera configured to be positioned at least partially within the expandable member.

Embodiment 2. The surgical stapler of Embodiment 1, wherein the insertion guide further comprises a tube extending from a proximal end of the expandable member, and wherein the tube comprises a first lumen and a second lumen extending therethrough.

Embodiment 3. The surgical stapler of Embodiment 2, wherein the first lumen is in fluid communication with an internal reservoir of the expandable member.

Embodiment 4. The surgical stapler of Embodiment 2 or 3, wherein the camera is configured to be positioned at least partially within the second lumen.

Embodiment 5. The surgical stapler of any one of Embodiments 2 to 4, wherein the first lumen terminates at a first opening positioned within the expandable member.

Embodiment 6. The surgical stapler of any one of Embodiments 2 to 5, wherein the second lumen terminates at a second opening positioned at a distal end of the tube.

Embodiment 7. The surgical stapler of any one of Embodiments 2 to 6, wherein the tube further comprises a first branch and a second branch positioned at a proximal end of the tube, wherein the first lumen extends through the first branch, and wherein the second lumen extends through the second branch.

Embodiment 8. The surgical stapler of Embodiment 7, wherein the insertion guide further comprises: a first port attached to the first branch and configured to attach to a fluid delivery device; and a second port attached to the second branch and configured to attach to a camera module.

Embodiment 9. The surgical stapler of Embodiment 8, wherein the tube further comprises a third lumen extending therethrough and a third branch positioned at the proximal end of the tube, and wherein the insertion guide further comprises a third port attached to the third branch and configured to attach to an air delivery device.

Embodiment 10. The surgical stapler of Embodiment 9, wherein the third lumen terminates at a third opening positioned at the distal end of the tube.

Embodiment 11. The surgical stapler of any one of Embodiments 1 to 10, wherein the expandable member comprises a balloon configured to radially expand and collapse to move the expandable member between the collapsed configuration and the expanded configuration.

Embodiment 12. The surgical stapler of any one of Embodiments 1 to 11, wherein the expandable member comprises a distal portion and a proximal portion, wherein the distal portion is configured to be positioned outside of the head assembly when the expandable member is in the expanded configuration, and wherein the proximal portion is configured to be positioned within the head assembly when the expandable member is in the expanded configuration.

Embodiment 13. The surgical stapler of Embodiment 12, wherein the distal portion has a partial egg shape, and wherein the proximal portion has a partial egg shape.

Embodiment 14. The surgical stapler of Embodiment 12 or 13, wherein the distal portion has a first maximum diameter, wherein the proximal portion has a second maximum diameter, and wherein the first maximum diameter is greater than the second maximum diameter.

Embodiment 15. The surgical stapler of any one of Embodiments 1 to 14, wherein the insertion guide is configured to extend through the tubular shaft and the head assembly.

Embodiment 16. A surgical stapler comprising: a tubular shaft having a proximal end and a distal end; a handle extending from the proximal end of the tubular shaft; a head assembly extending from the distal end of the tubular shaft; and an insertion guide configured to extend through the head assembly, the insertion guide comprising: (i) an expandable member configured to move between a collapsed configuration and an expanded configuration; and (ii) a tube extending from a proximal end of the expandable member, the tube comprising a first lumen and a second lumen extending therethrough.

Embodiment 17. The surgical stapler of Embodiment 16, wherein the first lumen is in fluid communication with an internal reservoir of the expandable member.

Embodiment 18. The surgical stapler of Embodiment 16 or 17, wherein the first lumen terminates at a first opening positioned within the expandable member.

Embodiment 19. The surgical stapler of any one of Embodiments 16 to 18, wherein the second lumen terminates at a second opening positioned at a distal end of the tube.

Embodiment 20. The surgical stapler of any one of Embodiments 16 to 19, wherein the tube further comprises a first branch and a second branch positioned at a proximal end of the tube, wherein the first lumen extends through the first branch, and wherein the second lumen extends through the second branch.

Embodiment 21. The surgical stapler of Embodiment 20, wherein the insertion guide further comprises: a first port attached to the first branch and configured to attach to a fluid delivery device; and a second port attached to the second branch and configured to attach to an air delivery device.

Embodiment 22. The surgical stapler of Embodiment 21, wherein the tube further comprises a third lumen extending therethrough and a third branch positioned at the proximal end of the tube, and wherein the insertion guide further comprises a third port attached to the third branch.

Embodiment 23. The surgical stapler of Embodiment 22, wherein the third lumen terminates at a third opening positioned at the distal end of the tube.

Embodiment 24. The surgical stapler of Embodiment 22 or 23, wherein the insertion guide further comprises a camera configured to be positioned at least partially within the third lumen.

Embodiment 25. The surgical stapler of Embodiment 24, wherein the camera is configured to be positioned at least partially within the expandable member.

Embodiment 26. The surgical stapler of any one of Embodiments 16 to 25, wherein the expandable member comprises a balloon configured to radially expand and collapse to move the expandable member between the collapsed configuration and the expanded configuration.

Embodiment 27. The surgical stapler of any one of Embodiments 16 to 25, wherein the expandable member comprises a distal portion and a proximal portion, wherein the distal portion is configured to be positioned outside of the head assembly when the expandable member is in the expanded configuration, and wherein the proximal portion is configured to be positioned within the head assembly when the expandable member is in the expanded configuration.

Embodiment 28. The surgical stapler of Embodiment 27, wherein the distal portion has a partial egg shape, and wherein the proximal portion has a partial egg shape.

Embodiment 29. The surgical stapler of Embodiment 27 or 28, wherein the distal portion has a first maximum diameter, wherein the proximal portion has a second maximum diameter, and wherein the first maximum diameter is greater than the second maximum diameter.

Embodiment 30. The surgical stapler of any one of Embodiments 16 to 29, wherein the insertion guide is configured to extend through the tubular shaft and the head assembly.

Embodiment 31. A surgical stapler system comprising: (i) a surgical stapler comprising: a tubular shaft having a proximal end and a distal end; a handle extending from the proximal end of the tubular shaft; a head assembly extending from the distal end of the tubular shaft; and an insertion guide configured to extend through the head assembly, the insertion guide comprising: an expandable member configured to move between a collapsed configuration and an expanded configuration; a camera configured to be positioned at least partially within the expandable member; and a tube extending from a proximal end of the expandable member, the tube comprising a first lumen, a second lumen, and a third lumen extending therethrough; (ii) a fluid delivery device configured to attach to the tube and be in fluid communication with the first lumen; (iii) a camera module configured to attach to the tube and be in operable communication with the camera; and (iv) an air delivery device configured to attach to the tube and be in fluid communication with the third lumen.

Embodiment 32. The surgical stapler system of Embodiment 31, wherein the tube further comprises a first branch, a second branch, and a third branch, wherein the first lumen extends through the first branch, wherein the second lumen extends through the second branch, and wherein the third lumen extends through the third branch.

Embodiment 33. The surgical stapler system of Embodiment 32, wherein the insertion guide further comprises: a first port attached to the first branch and configured to attach to the fluid delivery device; a second port attached to the second branch and configured to attach to the camera module; and a third port attached to the third branch and configured to attach to the air delivery device.

Embodiment 34. The surgical stapler system of any one of Embodiments 31 to 33, wherein the first lumen is in fluid communication with an internal reservoir of the expandable member.

Embodiment 35. The surgical stapler system of any one of Embodiments 31 to 34, wherein the first lumen terminates at a first opening positioned within the expandable member.

Embodiment 36. The surgical stapler system of any one of Embodiments 31 to 35, wherein the second lumen terminates at a second opening positioned at a distal end of the tube.

Embodiment 37. The surgical stapler system of any one of Embodiments 31 to 36, wherein the third lumen terminates at a third opening positioned at the distal end of the tube.

Embodiment 38. The surgical stapler system of any one of Embodiments 31 to 37, wherein the camera is configured to be positioned at least partially within the second lumen.

Embodiment 39. The surgical stapler system of any one of Embodiments 31 to 38, wherein the insertion guide further comprises a cable extending from a proximal end of the camera.

Embodiment 40. The surgical stapler system of any one of Embodiments 31 to 39, wherein the cable is configured to be positioned at least partially within the second lumen.

Embodiment 41. The surgical stapler system of any one of Embodiments 31 to 40, wherein the expandable member comprises a balloon configured to radially expand and collapse to move the expandable member between the collapsed configuration and the expanded configuration.

Embodiment 42. The surgical stapler system of any one of Embodiments 31 to 41, wherein the expandable member comprises a distal portion and a proximal portion, wherein the distal portion is configured to be positioned outside of the head assembly when the expandable member is in the expanded configuration, and wherein the proximal portion is configured to be positioned within the head assembly when the expandable member is in the expanded configuration.

Embodiment 43. The surgical stapler system of Embodiment 42, wherein the distal portion has a partial egg shape, and wherein the proximal portion has a partial egg shape.

Embodiment 44. The surgical stapler system of Embodiment 42 or 43, wherein the distal portion has a first maximum diameter, wherein the proximal portion has a second maximum diameter, and wherein the first maximum diameter is greater than the second maximum diameter.

Embodiment 45. The surgical stapler system of any one of Embodiments 31 to 44, wherein the insertion guide is configured to extend through the tubular shaft and the head assembly.

Embodiment 46. A method of introducing a surgical stapler into a patient, the method comprising: (i) advancing an insertion guide through a head assembly of the surgical stapler, such that the insertion guide extends distally beyond a distal end face of the head assembly; (ii) moving an expandable member of the insertion guide from a collapsed configuration to an expanded configuration; (iii) inserting the expandable member and the head assembly into a body cavity of the patient while the expandable member is in the expanded configuration; and (iv) capturing, via a camera of the insertion guide, video or image data of internal anatomy of the patient.

Embodiment 47. The method of Embodiment 46, wherein the camera is positioned at least partially within the expandable member.

Embodiment 48. The method of Embodiment 46 or 47, wherein the insertion guide further comprises a tube extending from a proximal end of the expandable member, and wherein the camera is positioned at least partially within a lumen of the tube.

Embodiment 49. The method of any one of Embodiments 46 to 48, wherein the expandable member comprises a balloon, and wherein moving the expandable member from the collapsed configuration to the expanded configuration comprises expanding the balloon.

Embodiment 50. The method of any one of Embodiments 46 to 49, wherein the surgical stapler further comprises a tubular shaft extending from a proximal end of the head assembly, and wherein the method further comprises advancing the insertion guide through the tubular shaft.

Embodiment 51. A method of introducing a surgical stapler into a patient, the method comprising: (i) advancing an insertion guide through a head assembly of the surgical stapler, such that the insertion guide extends distally beyond a distal end face of the head assembly; (ii) moving an expandable member of the insertion guide from a collapsed configuration to an expanded configuration; (iii) inserting the expandable member and the head assembly into a body cavity of the patient while the expandable member is in the expanded configuration; and (iv) delivering air through the insertion guide to insufflate the body cavity.

Embodiment 52. The method of Embodiment 51, wherein the insertion guide further comprises a tube extending from a proximal end of the expandable member, and wherein delivering the air through the insertion guide comprises delivering the air through a first lumen of the tube.

Embodiment 53. The method of Embodiment 51 or 52, wherein delivering the air through the insertion guide comprises delivering the air through the expandable member.

Embodiment 54. The method of any one of Embodiments 51 to 53, wherein the expandable member comprises a balloon, and wherein moving the expandable member from the collapsed configuration to the expanded configuration comprises expanding the balloon.

Embodiment 55. The method of any one of Embodiments 51 to 54, wherein the surgical stapler further comprises a tubular shaft extending from a proximal end of the head assembly, and wherein the method further comprises advancing the insertion guide through the tubular shaft.

Embodiment 56. A surgical stapler comprising: a tubular shaft having a proximal end and a distal end; a handle extending from the proximal end of the tubular shaft; a head assembly extending from the distal end of the tubular shaft; and an insertion guide which comprises: an expandable member configured to move between a collapsed configuration and an expanded configuration; and a tube extending from a proximal end of the expandable member, the tube comprising a first lumen and a second lumen extending therethrough, wherein the insertion guide is configured to be extended through the head assembly and then, with the expandable member in an expanded configuration, to facilitate introduction of the stapler into a patient's colon, and wherein the insertion guide, following introduction of the stapler into the patient's colon and subsequent transition of the expandable member into a collapsed configuration, is configured to be removed from at least the head assembly.

Embodiment 57. The surgical stapler of Embodiment 56, wherein the tubular shaft, the handle, and/or the head assembly comprises a lateral opening configured to allow the insertion guide to pass through the lateral opening and into a central bore of the head assembly during use of the insertion guide and to allow removal of the insertion guide from at least the central bore.

Embodiment 58. The surgical stapler of Embodiment 56 or 57, which is configured to be permit rapid removal and/or replacement of the insertion guide with the expandable member in a collapsed configuration.

Embodiment 59. The surgical stapler of any one of Embodiments 56 to 58, wherein the expandable member comprises a balloon configured to radially expand and collapse to transition between the collapsed configuration and the expanded configuration.

Embodiment 60. The surgical stapler of any one of Embodiments 56 to 59, wherein the tube further comprises a first branch and a second branch positioned at a proximal end of the tube, wherein the first lumen extends through the first branch, and wherein the second lumen extends through the second branch

Embodiment 61. The surgical stapler of any one of Embodiments 56 to 60, wherein the tube further comprises a first branch and a second branch positioned at a proximal end of the tube, wherein the first lumen extends through the first branch, and wherein the second lumen extends through the second branch.

Embodiment 62. The surgical stapler of Embodiment 61, wherein the insertion guide further comprises: a first port attached to the first branch and configured to attach to a fluid delivery device; and a second port attached to the second branch and configured to attach to a camera module.

Embodiment 63. The surgical stapler system of any one of Embodiments 56 to 62, wherein the expandable member comprises a distal portion and a proximal portion, wherein the distal portion is configured to be positioned outside of the head assembly when the expandable member is in the expanded configuration, and wherein the proximal portion is configured to be positioned within the head assembly when the expandable member is in the expanded configuration.

Embodiment 64. The surgical stapler system of Embodiment 63, wherein the distal portion has a partial egg shape, and wherein the proximal portion has a partial egg shape.

Modifications and variations of the devices, systems, and methods described herein will be obvious to those skilled in the art from the foregoing detailed description. Such modifications and variations are intended to come within the scope of the appended claims.

I claim:

1. A surgical stapler comprising:
a tubular shaft having a proximal end and a distal end;
a handle extending from the proximal end of the tubular shaft;
a head assembly extending from the distal end of the tubular shaft, the head assembly being configured to receive an anvil and comprising staples to be pressed against the anvil during stapling with the surgical stapler; and
an insertion guide configured to extend through the head assembly, the insertion guide comprising:
an expandable member defining an internal reservoir and being configured to move between a collapsed configuration and an expanded configuration in which at least part of the expandable member extends distally beyond a distal end face of the head assembly; and
a tube extending from a proximal end of the expandable member, the tube comprising a first lumen and a second lumen extending therethrough.

2. The surgical stapler of claim 1, wherein the first lumen is in fluid communication with the internal reservoir of the expandable member and terminates at a first opening positioned within the expandable member.

3. The surgical stapler of claim 2, wherein the second lumen terminates at a second opening positioned at a distal end of the tube.

4. The surgical stapler of claim 1, wherein the tube further comprises a first branch and a second branch positioned at a proximal end of the tube, wherein the first lumen extends through the first branch, and wherein the second lumen extends through the second branch.

5. The surgical stapler of claim 1, wherein the tube further comprises a third lumen extending therethrough and the third lumen terminates at a third opening positioned at the distal end of the tube.

6. The surgical stapler of claim 5, wherein the insertion guide further comprises a camera configured to be positioned at least partially within the third lumen.

7. The surgical stapler of claim 6, wherein the camera is configured to be positioned at least partially within the expandable member.

8. The surgical stapler of claim 1, wherein the expandable member comprises a balloon configured to radially expand and collapse to move the expandable member between the collapsed configuration and the expanded configuration.

9. The surgical stapler of claim 1, wherein the expandable member comprises a distal portion and a proximal portion, wherein the distal portion is configured to be positioned outside of the head assembly when the expandable member is in the expanded configuration, and wherein the proximal portion is configured to be positioned within the head assembly when the expandable member is in the expanded configuration.

10. The surgical stapler of claim 1, wherein the insertion guide is configured to extend through the tubular shaft and the head assembly.

11. The surgical stapler of claim 1, wherein the expandable member in the expanded configuration has an outer surface curved to facilitate atraumatic insertion of the expandable member and head assembly into a patient's colon.

12. A surgical stapler comprising:
a tubular shaft having a proximal end and a distal end;
a handle extending from the proximal end of the tubular shaft;
a head assembly extending from the distal end of the tubular shaft, the head assembly being configured to receive an anvil and comprising staples to be pressed against the anvil during stapling with the surgical stapler; and
an insertion guide which comprises:
an expandable member defining an internal reservoir, the expandable member being configured to move between a collapsed configuration and an expanded configuration; and
a tube extending from a proximal end of the expandable member, the tube comprising a first lumen and a second lumen extending therethrough,
wherein the insertion guide is configured to be extended through the head assembly and then, with the expandable member in an expanded configuration, to facilitate introduction of the head assembly and at least part of the tubular shaft into a patient's colon, and
wherein, following introduction of the head assembly and at least part of the tubular shaft into the patient's colon and subsequent transition of the expandable member into a collapsed configuration, the insertion guide is configured to be removed from at least the head assembly.

13. The surgical stapler of claim 12, wherein the tubular shaft, the handle, and/or the head assembly comprises a lateral opening configured to allow the insertion guide to pass through the lateral opening and into a central bore of the head assembly during use of the insertion guide and to allow removal of the insertion guide from at least the central bore.

14. The surgical stapler of claim 12, which is configured to be permit rapid removal and/or replacement of the insertion guide with the expandable member in a collapsed configuration.

15. A method of introducing a surgical stapler into a patient, the method comprising:
advancing an insertion guide through a head assembly of the surgical stapler, such that a distal end portion of the insertion guide extends distally beyond a distal end face of the head assembly;
expanding an expandable member of the distal end portion of the insertion guide from a collapsed configuration to an expanded configuration by expanding an internal reservoir of the expandable member;
inserting the expandable member together with the head assembly into a body cavity of the patient while the expandable member is in the expanded configuration; and
after the inserting, collapsing the expandable member and retracting the insertion guide through the head assembly.

16. The method of claim 15, further comprising capturing, via a camera of the insertion guide, video or image data of internal anatomy of the patient.

17. The method of claim 16, wherein the camera is positioned at least partially within the expandable member.

18. The method of claim 15, wherein the expandable member comprises a balloon, and wherein expanding the expandable member from the collapsed configuration to the expanded configuration comprises expanding the balloon.

19. The method of claim 15, wherein the surgical stapler further comprises a tubular shaft extending from a proximal end of the head assembly, and wherein the method further comprises advancing the insertion guide through the tubular shaft.

20. The method of claim 15, further comprising delivering air through the insertion guide to insufflate the body cavity, wherein delivering the air through the insertion guide optionally comprises delivering the air through the expandable member.
